(19) United States
(12) Patent Application Publication
(10) Pub. No.: US 2017/0245779 A1
(43) Pub. Date: Aug. 31, 2017

(54) PRESSING APPARATUS FOR MAGNETIC RESONANCE IMAGING AND SPECTROSCOPY

(71) Applicants: NATIONAL RESEARCH COUNCIL OF CANADA, Ottawa (CA); SUNNYBROOK RESEARCH INSTITUTE, Toronto (CA)

(72) Inventors: Masoom HAIDER, Toronto (CA); Scott B. KING, Winnipeg (CA); Mike SMITH, Winnipeg (CA); Jarod MATWIY, Winnipeg (CA); John Calvin BEWSKY, Winnipeg (CA); Hung Yu LIN, Winnipeg (CA)

(21) Appl. No.: 15/519,747

(22) PCT Filed: Oct. 16, 2015
(86) PCT No.: PCT/CA2015/051041

§ 371 Date: Apr. 17, 2017

Related U.S. Application Data
(60) Provisional application No. 62/065,424, filed on Oct. 17, 2014.

(51) Int. Cl. A61B 5/055; G01R 33/567; G01R 33/3415; G01R 33/30; G01R 33/34

(52) U.S. Cl. CPC A61B 5/0555; G01R 33/30; G01R 33/34007; G01R 33/3415; G01R 33/5673

(57) ABSTRACT

A magnetic resonance (MR) apparatus, the MR apparatus including a tissue pressing surface having one or more MR coil elements, the pressing surface being configured and sized to selectively indent a portion of an exterior tissue of a subject when a physical force is applied to the indenting substrate thereby reducing a distance between the one or more MR coil elements and a structure of interest located inside the subject.

### FIELD OF THE INVENTION

The present invention relates in general to magnetic resonance imaging and spectroscopy and devices related thereto, in particular to methods and devices used to enhance signal to noise ratio and obtain high-quality magnetic resonance spectra, and more particularly to devices that reduce the distance between a MR coil and a structure of interest inside a subject.

### BACKGROUND OF THE INVENTION

Magnetic resonance imaging (MRI) and spectroscopy (MRS) require high signal to noise ratio (SNR) to obtain high-quality magnetic resonance (MR) spectra and images. For a typical surface coil, signal drop-off inversely follows with distance squared, making difficult to achieve high SNR for MRI/MRS applications on anatomical regions that are not superficial. In some instances, internal coils have been utilized (intra-vascular/endorectal coils), but have limited applications and are often uncomfortable for patients. External surface coils for these non-superficial regions are designed using large coils to have best possible signal sensitivity at the required depths. There are multiple regions of clinical significance that are not suited for internal coil applications and would greatly benefit from improved SNR over what can be achieved using typical external surface coils. The diagnostic capability of MRI/MRS for external surface coil applications to internal organs/tissue of interest such as pancreas MRI could be improved.

Presently, to obtain MRI/MRS images or spectra from a structure that is located within a subject, a surface loop coil is used to produce maximum SNR at a particular depth of interest within a subject or sample. The size and geometry (square, circular, etc) of the loop coil is improved for that depth of interest. A second, so called “butterfly” coil element may be added on top of the loop coil and combined into a single channel using an improved combiner to increase the SNR at that depth of interest typically by 20-30% [1]. For that depth of interest, this technique is thought to increase the SNR for a single channel. To increase fields-of-view (FOV) in the long dimension additional inductively decoupled loop-butterfly elements are placed along the long axis and fed into separate receivers. Alternatively, a phased array of surface loop coils can be employed, but like the standard single surface coil, the SNR is ultimately limited by the distance between the surface coil or array and the depth of interest inside the body of the subject. Alternative approaches for internal structures include, as mentioned above, internal coils, such as endorectal [2] or intravascular [3]. Furthermore, internal coils have limited applications as the coils have to be very small to accommodate being inserted into the patient. This results in localized and inhomogeneous sensitivity maps. Internal coils are not a practical consideration for many internal structures such as the pancreas or kidneys. The use of endocrectal coils for prostate diagnosis also has complications of patient discomfort and significant tissue deformation [4].

U.S. Pat. No. 8,380,281 describes a compression device for enhancing normal/abnormal tissue contrast in MRI. The compression device has a fixed surface and a moveable member. The target tissue to be compressed is located between the fixed surface and the movable member. When the movable member is moved the target tissue is compressed between the fixed surface and the movable member. The compression device of this US patent is suitable for compression of an entire breast of the subject while the breast is disposed within a conventional MRI breast coil, which may result in a reduction of comfort to the subject. Furthermore, the “target” in U.S. Pat. No. 8,380,281 is the tissue (i.e. the breast) as opposed to a structure inside the body/breast of the subject. The devices and methods of U.S. Pat. No. 8,380,281 only work with target tissues that extend from the body, as such the devices and methods of this patent will not work for structures of interest inside a subject, for example, inside the abdomen. Furthermore, the device of U.S. Pat. No. 8,380,281 does not include a MR coil in the movable compression member.

In view of the foregoing, there is a need for devices or methods that will reduce the distance between the MR coil and a structure of interest inside a body of an object or subject thereby enabling improved SNR behavior from the MR coil. There is also a need that the device and method for reducing the distance between the MR coil and a structure of interest does not result in patient discomfort.

### SUMMARY OF THE INVENTION

Presented herein are devices and methods that selectively press a portion of an area of a subject or object so as to bring a magnetic resonance (MR) coil closer to a structure of interest inside the subject or object by displacing tissue and/or organs that may be in between the devices of the present invention and the structure of interest. Bringing the MR coil closer to an internal structure of interest enables improved signal to noise ratio (SNR) behavior from the MR coil. Selectively pressing a smaller surface than the entire breast or abdomen, for example, will bring MR coils within the device closer to the desired internal structure inside the breast or abdomen, than could be achieved with a plate that compresses the entire breast or abdomen. That said, the pressing surface of the present invention, selectively presses or indents a surface area that is generally smaller than the surface area of the abdomen, breast, leg, neck of the subject.

Conversely, the methods and compression-type devices such as that of U.S. Pat. No. 8,380,281 do not selectively press (i.e. does not displace tissue out of the way) a portion of the breast, but rather it compresses an entire breast.

In one embodiment, the present invention provides for a magnetic resonance (MR) apparatus. The MR apparatus of the present invention, in one embodiment, includes a tissue pressing surface and one or more MR coil elements coupled to the tissue pressing surface, the pressing surface being configured and sized to selectively press a portion of an exterior tissue of a subject when a physical force is applied to the pressing surface thereby reducing a distance between the one or more MR coil elements and a structure of interest located inside the subject.

In another embodiment of the MR apparatus of the present invention, the MR apparatus includes a top surface opposite to the tissue pressing surface, and wherein the one or more MR coil elements are housed in between the tissue pressing surface and the top surface.

In another embodiment of the MR apparatus of the present invention, the MR apparatus further includes one or more secondary devices capable of mating to the pressing surface, each secondary device having one or more additional MR coil elements.

In another embodiment of the MR apparatus of the present invention, the MR apparatus includes one secondary device, and the one secondary device has a periphery larger than a periphery of the pressing surface.

In another embodiment of the MR apparatus of the present invention, the MR apparatus includes more than one secondary device and the more than one secondary devices are capable of mating to the pressing surface around a periphery of the pressing surface.

In another embodiment of the MR apparatus of the present invention, the MR apparatus further includes a member coupling having an end attached to the pressing surface and a free end.

In another embodiment of the MR apparatus of the present invention, the pressing surface includes a member coupling having an end attached to the pressing surface and a free end, and at least one of the one or more secondary devices includes an engaging means for receiving the free end of the member coupling.

In another embodiment of the MR apparatus of the present invention, the engaging means has a channel extending through the at least one of the one or more secondary devices.

In another embodiment of the MR apparatus of the present invention, the pressing surface includes a member coupling having an end attached to the top surface and a free end, the MR apparatus includes two or more secondary devices, and the two or more secondary devices are modular pieces that fit together to create an engaging means for receiving the free end of the member coupling.

In another embodiment of the MR apparatus of the present invention, the member coupling includes a hollow interior configured to house electronic components of the one or more coil elements coupled to the pressing surface.

In another embodiment of the MR apparatus of the present invention, the MR apparatus further includes a means for applying a physical force to the pressing surface.

In another embodiment of the MR apparatus of the present invention, the means for applying physical force is capable of attachment to the free end of the member coupling.

In another embodiment of the MR apparatus of the present invention, the MR apparatus further includes a driving means operatively linked to the means for applying physical force.

In another embodiment of the MR apparatus of the present invention, the subject has limbs and the means for applying a physical force to the pressing surface includes limb support configured to receive the limbs of the subject.

In another embodiment of the MR apparatus of the present invention, the subject is an animal and the one or more secondary devices are sized to cover a torso of the animal.

In another embodiment of the MR apparatus of the present invention, the one or more coil elements of the pressing surface and the additional one or more coil elements of the secondary devices include MR signal receiving coils, RF transmitting coils, gradient coils or any combination thereof.

In another embodiment of the MR apparatus of the present invention, the additional one or more coil elements of the one or more secondary devices are laid out within the secondary devices so as to reduce electromagnetic coupling with the one or more coil elements of the pressing surface when the one or more secondary devices is/are mated to the indenting substrate.

In another embodiment of the MR apparatus of the present invention, the subject is a human and the MR apparatus further includes a means for use by the human subject to apply a physical force to the pressing surface, and wherein the means for applying physical force is capable of attaching or coupling to the pressing surface.

In another embodiment of the MR apparatus of the present invention, the means for use by the human subject to apply physical force to the pressing surface includes limb support configured to receive limbs of the human.

In another embodiment of the MR apparatus of the present invention, the one or more coil elements of the pressing surface includes MR signal receiving coils, RF transmitting coil, gradient coils or any combination thereof.

In another embodiment of the MR apparatus of the present invention, the tissue pressing surface is made of a substantially rigid material.

In another embodiment of the MR apparatus of the present invention, the pressing surface has a relatively thin profile.

In another embodiment of the MR apparatus of the present invention, the subject is an animal, and the pressing surface is sized to cover an abdominal portion, a chest portion of, a neck portion or a limb of the animal.

In another embodiment of the MR apparatus of the present invention, the subject is an animal and the structure of interest is an organ or a tumor. In one aspect of this embodiment the subject is a human.

In another embodiment of the MR apparatus of the present invention, the subject is a vertebrate and the structure of interest includes an organ of a skeletal system, endocrine system, urogenital apparatus, digestive apparatus, respiratory apparatus, circulatory system or nervous system, synovial fluid, blood and/or lymph of the vertebrate.

In another embodiment of the MR apparatus of the present invention, the subject is a mammal, and the structure of interest is inside a breast of the mammal.

In another embodiment of the MR apparatus of the present invention, the subject is a human and the pressing surface is configured to press a breast of the human in the prone position.

In another embodiment of the MR apparatus of the present invention, the subject is a MR phantom and the exterior tissue is an exterior surface of the phantom.

In another embodiment of the MR apparatus of the present invention, the subject is a plant, and the exterior tissue is an epidermis of the plant.

In another embodiment of the MR apparatus of the present invention, the subject is an animal, and the exterior tissue is an epidermis of the animal.

In another embodiment of the MR apparatus of the present invention, the subject is an organ or a tissue biopsy.

In another embodiment of the MR apparatus of the present invention, the subject is a vertebrate, and the pressing surface includes a convex curved portion configured to accommodate an inferior curved costal margin of a rib cage of the subject, or configured to accommodate a curvature formed by a pubic bone and both superior pubic rami, or formed to fit inferior to an abdominal pannus over the pubic bone.

In another embodiment of the MR apparatus of the present invention, the pressing surface comprises a substantially rigid material having a thin profile, the pressing surface comprising a first wing region, a tail region, a head region, and a second wing region, the first wing region and second wing region extending longitudinally away from a midline connecting the head and tail regions, the head region being defined by a convex shape and the tail region being defined by a concave shape.

In another embodiment of the MR apparatus of the present invention, the one or more MR coil elements contour the periphery of the pressing surface.

In another embodiment of the MR apparatus of the present invention, the first and second wing regions curve from the midline such that the subject contacting side of the MR apparatus is capable of approximately conform to the shape of the exterior tissue of the subject.

In another embodiment of the MR apparatus of the present invention, the one or more MR coil elements are coated with a suitable coating, and the combination of each of said one or more MR coil elements with the coating constitutes the pressing surface.

In another embodiment, the present invention is a method of recording magnetic resonance (MR) signals from a structure of interest inside a subject. The method, in one embodiment, includes: (a) providing a MR apparatus having a tissue pressing surface and one or more MR signal receiving coils coupled to the tissue pressing surface, the tissue pressing surface being configured and sized to selectively press a portion of an exterior tissue of a subject; (b) positioning the pressing surface in contact with a portion of the subject's exterior tissue that is relatively near to where the structure of interest is located inside the subject; (c) exposing the portion of the subject's exterior tissue to an MR system having a Larmor frequency; (d) stimulating the structure of interest at the Larmor frequency of the MR system; and (e) receiving MR signals from the stimulated structure of interest with the one or more MR receiving coils of the MR apparatus.

In one embodiment of the method of recording MR signals from the structure of interest inside the subject, prior to step (d) the method further includes the step of applying physical force to the pressing surface thereby selectively pressing the portion of the subject's exterior tissue and reducing a distance between the one or more MR signal receiving coils of the MR apparatus and the structure of interest inside the subject.

In one embodiment of the method of recording MR signals from a structure of interest inside a subject, the MR apparatus further includes one or more transmitting coils capable of transmitting signals at the Larmor frequency of the MR system, and wherein the stimulation of the structure of interest is done with the transmitting coils of the MR apparatus.

In another embodiment of the method of recording MR signals from a structure of interest inside a subject, the MR apparatus further includes one or more gradient coils, and wherein the method further includes performing variations in a main magnetic field (Bo) of the MR system with the gradient coils.

In another embodiment of the method of recording MR signals from a structure of interest inside a subject, the MR apparatus is the MR apparatus of any one of embodiments of the present invention.

In another embodiment, the present invention is a method of transmitting signals at the Larmor frequency of a magnetic resonance (MR) system to a structure of interest inside a subject. In one embodiment, the method of transmitting signals at the Larmor frequency of a magnetic resonance (MR) system to a structure of interest inside a subject includes: (a) providing a MR apparatus having a tissue pressing surface and one or more transmitting coils coupled to the tissue pressing surface, the tissue pressing surface being configured and sized to selectively press a portion of an exterior tissue of a subject; (b) positioning the pressing surface in contact with a portion of the subject's exterior tissue that is relatively near to where the structure of interest is located inside the subject; (c) exposing the portion of the subject's exterior tissue to an MR system having a Larmor frequency; and (d) transmitting the signal at the Larmor frequency with the one or more transmitting coils thereby stimulating the structure of interest.

In one embodiment of the method of transmitting signals at the Larmor frequency of the MR system to the structure of interest of the present invention, prior to step (c) the method further includes the step of applying physical force to the pressing surface thereby selectively pressing the portion of the exterior tissue and reducing a distance between the one or more transmitting coils and the structure of interest.

In one embodiment of the method of transmitting signals at the Larmor frequency of a MR system to a structure of interest inside a subject of the present invention, the MR apparatus further includes MR signal receiving coils, and wherein the method further includes the step of receiving MR signals from the stimulated structure of interest with the one or more MR signal receiving coils of the MR apparatus.

In another embodiment of the method of transmitting signals at the Larmor frequency of a MR system to a structure of interest inside a subject of the present invention, the MR apparatus further includes one or more gradient coils, and wherein the method further includes performing variations in a main magnetic field (Bo) of the MR system with the gradient coils.

In another embodiment of the method of transmitting signals at the Larmor frequency of a MR system to a structure of interest inside a subject of the present invention, the MR apparatus is the MR apparatus of any one of embodiments of the present invention.

In one embodiment of the method of recording MR signals or transmitting MR signals of the previous embodiments, the subject is a human and the step of applying the physical force includes: (i) having the subject produce an inhalation stroke; (ii) applying the physical force to the pressing surface during the inhalation stroke; (iii) holding the pressing surface for a sufficient amount of time while the subject keeps the inhalation stroke; (iv) having the subject exhale and release the pressing surface; and (v) repeating sub-steps (i)-(v) as necessary.

In yet another embodiment, the present invention is a use of a magnetic resonance (MR) apparatus having one or more MR coil elements and a tissue pressing surface to record, transmit or to record and transmit MR signals form a structure of interest inside a subject. In one aspect of this embodiment, the MR apparatus is the MR apparatus of any of the embodiments of the present invention.

In a further embodiment, the present invention is a magnetic resonance (MR) apparatus. The MR apparatus, in one embodiment, includes a surface having one or more MR coil elements, the surface comprising a first wing region, a tail region, a head region, and a second wing region, the first wing region and second wing region extending longitudinally away from a midline connecting the head and tail regions, the head region being defined by a convex shape and the tail region being defined by a concave shape.

In one embodiment of the MR apparatus of the previous embodiment, the one or more MR coil elements contour a periphery of the indenting substrate.

In another embodiment of the MR apparatus of the previous two embodiments, the first and second wing regions curve from the midline such that the subject contacting side of the MR apparatus is capable of approximately conforming to the shape of the exterior tissue of the subject or body part.

In another embodiment, the present invention is a magnetic resonance (MR) apparatus, the MR apparatus including a tissue pressing surface and one or more MR coil elements coupled to the pressing surface, the pressing surface being configured and sized to selectively press a portion of an exterior tissue of a body part an animal when a physical force is applied to the pressing surface thereby reducing a distance between the one or more MR coil elements coupled to the surface and a structure of interest located inside the subject, wherein the size of the portion ranges between about 1/20 to about ⅔ of the exterior tissue of the body part.

In one embodiment the body part is an abdomen, and the size of the portion ranges between 1/20 to about ⅔ of the exterior tissue of the abdomen.

In another embodiment the body part is a breast, and the size of the portion ranges between 1/20 to about ½ of the exterior tissue of the breast.

The present invention, in yet another embodiment, provides for a magnetic resonance (MR) apparatus, as substantially described herein with reference to and as illustrated by the accompanying drawings.

The present invention, in yet a further embodiment, provides for a method of recording or transmitting a magnetic resonance (MR) signal from or to a structure of interest inside a subject as substantially described herein with reference to and as illustrated by the accompanying drawings.

### BRIEF DESCRIPTION OF THE DRAWINGS

The following figures illustrate various aspects and preferred and alternative embodiments of the invention.

FIG. 1: Graph illustrating a perspective view from the back side of a pressing device in accordance to one embodiment of the present invention.

FIG. 2: Graph illustrating a top plan view of the pressing device of FIG. 1.

FIG. 3: Graph illustrating a front view of the pressing device of FIG. 1.

FIG. 4: Graph illustrating a side view of the pressing device of FIG. 1.

FIG. 5: Not to scale circuit board computer aided design layout for a 5 channel pressing pad.

FIG. 6: Not to scale circuit board computer aided design layout for an 8 channel secondary torso coverage in accordance to one embodiment of the present invention.

FIG. 7: a) Photograph of a close-up overhead view of a 5 channel pressing device coil array prototype designed for 3T magnetic resonance imaging in accordance to one embodiment of the present invention; b) Photograph of an angled view of the prototype of panel a) to show the output cable and curved profile of the housing; c) Outer or secondary device 8-channel array prior to electrical construction.

FIG. 8: Graph illustrating a front perspective view of a MR apparatus of the present invention with the pressing surface in a non-pressing position.

FIG. 9: Graph illustrating a front perspective view of the MR apparatus of FIG. 8 with the pressing surface in the pressing position.

FIG. 10: Graphs illustrating an example of a method of recording and/or transmitting magnetic resonance (MR) signals from and/or to a structure of interest inside a subject using a MR apparatus of the present invention. A: exploded, cross sectional view (Z axis) of a MR apparatus of the present invention. B: assembled MR apparatus of panel A in a non-pressing position. C: Assembled MR apparatus of panel B positioned on a portion of an exterior tissue of a subject relatively near a structure of interest. D: Assembled MR apparatus of panel A in a pressing position. E: Assembled MR apparatus of panel D pressing on a portion of an exterior tissue of a subject relatively near to a structure of interest.

FIG. 11: Graph illustrating a bottom perspective view of a MR apparatus being positioned on a subject according to one embodiment of the present invention.

FIG. 12: Graph illustrating a side view of a MR apparatus on a subject according to one embodiment of the present invention.

FIG. 13: Graph of an MR apparatus of the present invention illustrating a secondary device modules split apart according to one embodiment of the present invention.

FIG. 14: Graph of the MR apparatus of FIG. 13 illustrating the secondary substrate modules fit together.

FIG. 15: Photograph illustrating three iterations of the trace layout for the 8 channel torso coverage circuit board. Mirror image symmetry was employed along the central S-I plane of the design.

FIG. 16: Illustration of a 14 channel phased array coil design, based on an 11 channel concentric anterior array with 3 channel Original Equipment Manufactured (OEM) posterior array. Shown is a) non-pressed model with coil, b) 0 cm—non-pressed meshed, c) 2 cm—lightly pressed mesh, and d) 4 cm—heavily pressed mesh.

FIG. 17: Simulated SNR ratio maps comparing the pressed models illustrated in FIG. 14 to the baseline (0 cm) for both axial slices. Averaged values are displayed on a 8×8 grid, where the imaging FOV was 32 cm×24 cm (L-R×A-P).

FIG. 18: Photographs illustrating materials used for designing the pressing surface of the present invention, including a) a variety of shapes, sizes and thicknesses of balsa wood pads to enable the pressing. b) The underside of the rigid plastic former shows a mechanism for varying the structure of the pressing pad. c) An option with a guide to hold the belt used for pressing leverage.

FIG. 19: Axial images of the abdomen of a 100 Kg adult male without pressing. A: inhale; B: exhale.

FIG. 20: Axial MRI slices of the abdomen for a 100 Kg adult male on inhale with pressing (A) and without pressing (B).

FIG. 21: Sagittal MRI slices of the abdomen for a 100 Kg adult male on inhale with pressing (A) and without pressing (B).

FIG. 22: Axial MRI slices for a 100 Kg adult male on inhale with pressing (A) and without pressing (B).

FIG. 23: Sagittal MRI slices for a 100 Kg adult male on inhale with pressing (A) and without pressing (B).

FIG. 24: Photographs of MRI phantom used to mimic the torso of a subject and enable comparisons between a) non-pressed and b) pressed states.

FIG. 25: Graph illustrating noise correlation properties for the 5 channel pressing plate coil, showing a) normalized noise correlation, b) non-normalized values and c) phase components.

FIG. 26: Graphs illustrating Non-pressing SNR, considering a 10 cm deep coronal slice for a) 6 channel (Ch) OEM coil, b) 11Ch (6 Ch OEM and 5 Ch indentation array), and c) SNR ratio depicting improvement from the pressing plate without indentation. d) shows the non-pressing SNR gains using the 5 Ch pressing array for a central axial slice. The dashed oval represents the pancreas region.

FIG. 27: Graphs comparing the non-pressing to 5 cm pressing SNR maps for a 10 cm deep coronal slice, displaying a) 6 Ch OEM without pressing, b) 11Ch (6 Ch OEM and 5 Ch pressing array) with pressing, and c) SNR ratio depicting improvements from the pressing array with pressing. The dashed oval represents the pancreas region.

FIG. 28: Graphs comparing the non-pressing to 5 cm pressing SNR maps for a central axial slice, displaying a) 6 Ch OEM without pressing, b) 11 Ch (6 Ch OEM and 5 Ch pressing array) with indentation, and c) SNR ratio depicting improvements from the pressing array with indentation. The dashed oval represents the pancreas region.

FIG. 29: Graph illustrating an example of a method of recording and/or transmitting magnetic resonance (MR) signals from and/or to a structure of interest inside a subject using a MR apparatus of the present invention.

FIG. 30: Sagittal MRI slice of the abdomen a healthy human using the apparatus of FIGS. 10 and 11.

FIG. 31: Transverse slice of the abdomen of a healthy human using the OEM RF coil.

FIG. 32: Transverse slice of the abdomen of a healthy human using the apparatus of FIGS. 10 and 11.

FIG. 33: Signal to Noise Ratio (SNR) maps for the pressing array (panel A) and OEM array (panel B).

FIG. 34: g-factor maps for the pressing array (panel A) and OEM array (panel B).

### DESCRIPTION OF THE INVENTION

### Definitions

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. In order to aid in the understanding and preparation of the within invention, the following illustrative, non-limiting, examples are provided. The priority document is incorporated by reference to this document.

“Exterior tissue” refers in this document to an outermost layer of a subject or the outmost layer of a body part. The “exterior tissue” may depend on the nature of the subject or body part. In the case of an animal or plant, the exterior tissue will be the skin of the animal or the epidermis of a plant and it will not include endothelium, or the tissue lining the inside cavities, tracts and lumen of the animal or plant; in the case of a body part or body section (i.e. the abdomen, breast, neck, limb and so forth) “exterior tissue” will be the skin covering the body part or body section; in the case of an ex vivo organ or tissue biopsy obtained from an animal or plant, the “exterior tissue” will be the layer of cells lining the exterior of the organ or biopsy; and in the case of a MR phantom, the “exterior tissue” refers to the outermost layer of the MR phantom. Preferably, the exterior tissue is an indentable tissue.

“Pressing” or “press” refers in this document to the pressing of a surface or substrate onto a portion of exterior tissue of a region or part of a subject or object, such that the surface penetrates, including indents, into the subject or object but without breaking or piercing the exterior tissue, or without leaving a permanent mark or permanent dent on the exterior tissue. The size of the portion of exterior tissue may depend on the region or part of the subject or object, and it may range from about 1/20 to about ⅔, but less than 1/20 and more than ⅔ may also be possible. For example, “pressing a portion of the abdomen” refers to pressing a surface onto a portion of the abdomen (the anterior portion of the body of a vertebrate between the thorax and the pelvis), and not to the entire abdomen, for example, pressing on 1/20, 1/10, ¼, ⅓, ½, ⅔ and so forth of the abdomen area and including any area there in between; and “pressing a breast” refers to pressing a surface onto a relatively small portion of the breast, but not the entire breast, for example pressing on 1/20, 1/10, ¼, ⅓, ½ and so forth of the breast's area including any area there in between. The smaller the portion being pressed the deeper the press. “Pressing” is not the same as “compression,” which refers to the non-selective pressing of a surface onto a relatively large tissue portion of or the entire region or part of a subject so as to reduce a volume of the tissue portion being compressed.

The term “pressing surface” refers in this document to a material-carrying MR coil which is used to apply a physical force to a portion of an exterior tissue of a subject or object so as to bring the MR coil being carried by the material relatively closer to a target located under the exterior tissue or object (i.e. inside the subject or object). At the same time the MR coil may transmit or receive MR signal data from, or deliver gradient fields to, the target and the rest of the body. “Pressing surface” excludes inserting or implanting the MR coil under the exterior tissue or inside the body of the subject or object. The material may be a shell or pad carrying the MR coil(s) or the material may be a coating on an MR coil.

“Subject” refers in this document to subjects, objects and to any matter or body from which an MR signal can be recorded. Examples of subjects include living objects, such as plants, human and non-human animals, as well as phantoms designed for MR experiments (MR phantoms). The term “subject” also includes samples obtained or extracted from the subject, such as organs and/or biopsies.

“Structure” refers to any matter or body inside the subject from which a MR signal can be recorded. Examples of structures may include the organs of an animal (pancreas, stomach, liver, intestines, kidneys, prostate, uterus, bladder, and so forth). “Structure” may also be used to refer to a mass inside a subject such as a tumor, or to fluids, such as blood, lymphatic and synovial fluids. Preferably, the structure may be located generally near tissue capable of being pressed, such as the abdomen or the breast of an animal. Structure of interest may be a structure or an exploratory area of imaging below the exterior tissue.

Presented herein is an MR apparatus having an exterior tissue pressing surface carrying one or more MR coil elements.

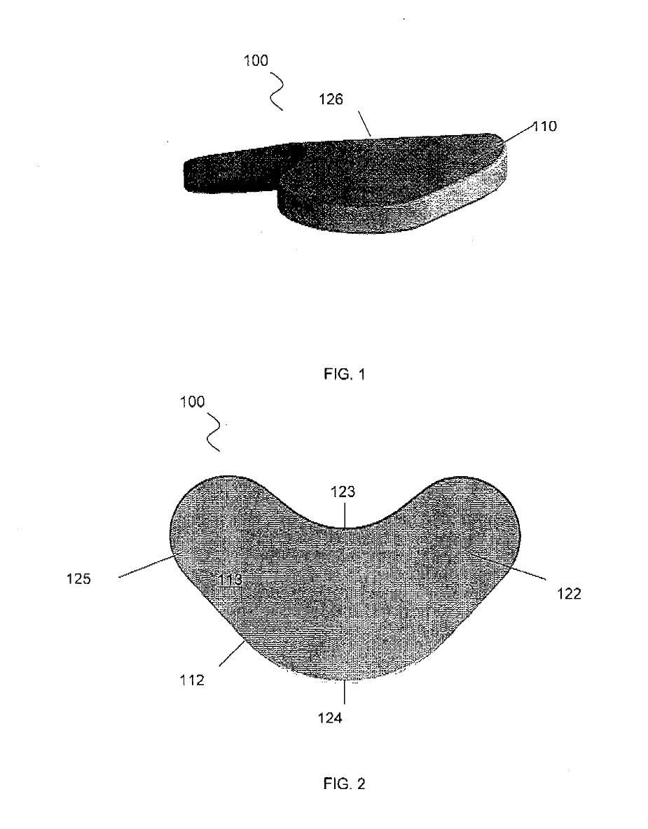

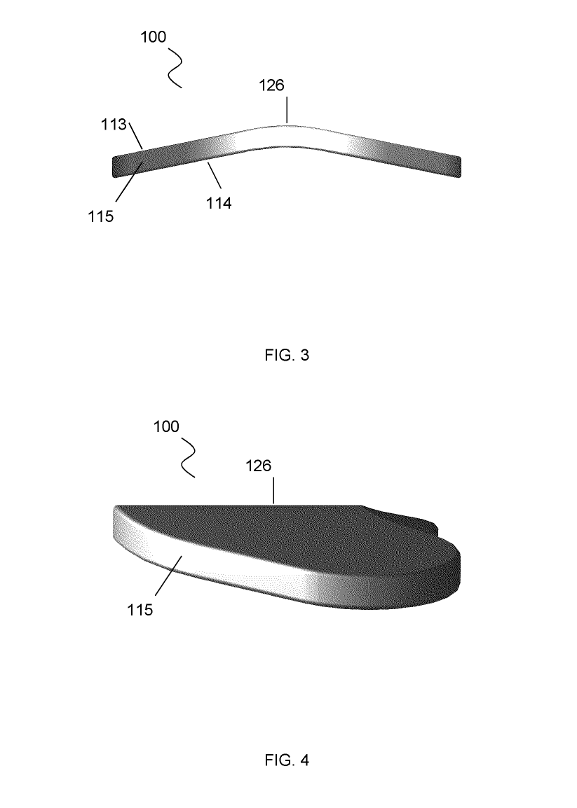

FIGS. 1-5 illustrate an MR apparatus 100 according to one embodiment of the present invention. The MR apparatus includes a tissue pressing surface 114. The pressing surface may have or carry one or more MR coil elements 120. The coil elements may be coupled or attached or fixed to the pressing surface or they may be housed within the pressing surface 114 and an opposite surface 113. In another embodiment of the present invention, the one or more MR coil elements may be coated with a suitable coating, such as an insulator. In this embodiment, the pressing surface may be the coated MR coil elements.

Referring to FIGS. 1-5 and 7 the MR apparatus 100 may be described as a device 110 having a periphery 112, and surfaces 113 and 114. For convenience one surface may be referred to as a top surface 113 and the other surface as a bottom surface or pressing surface 114. The bottom surface may also be referred to as the subject-contacting surface. The top 113 and bottom 114 surfaces may define an interior chamber or casing 116 (shown in FIG. 7a). To form the casing the top and bottom sides 113, 114 may be connected at their peripheral ends to side walls 115. Alternatively, the top and bottom sides may be connected at their peripheral ends throughout a periphery of the pressing surface (not shown in the figures). The side walls 115 may extend between the top 113 and bottom sides 114 thereby forming an interior casing 116 (shown in FIG. 7a). In one embodiment, casing 116 may serve to house the coil elements. In the embodiment illustrated in the figures, the one or more coil elements 120 may be housed within the interior casing 116. In another embodiment, not shown in the figures, the MR apparatus may be a substantially solid substrate or pad having a first, subject-contacting, pressing surface and a second surface, and the MR coils may sit on, be coupled to or fixed to the second surface. In another embodiment, the one or more MR coil elements may be coated with a suitable coating, such as an insulator, and thus the subject contacting surface is the coating.

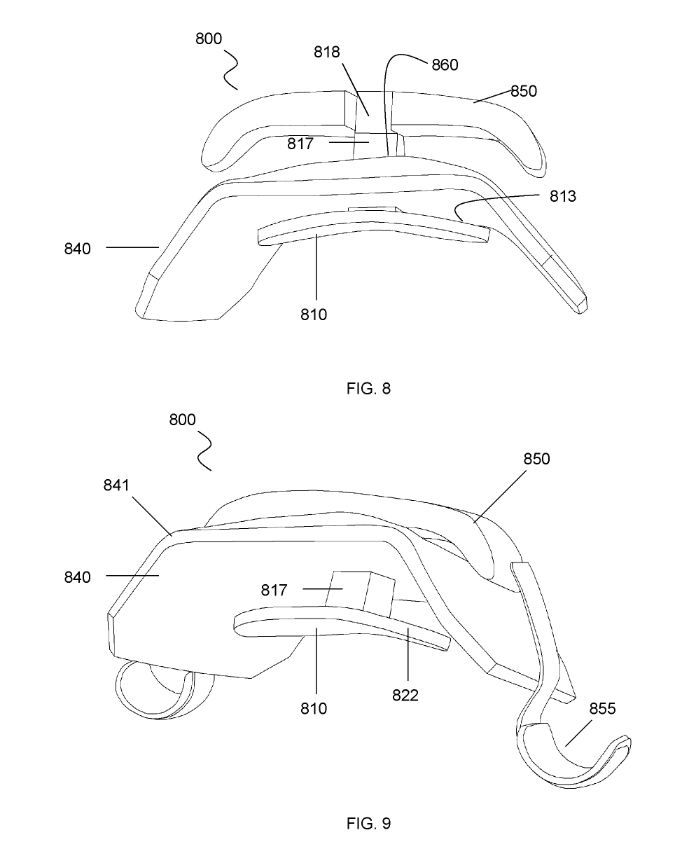

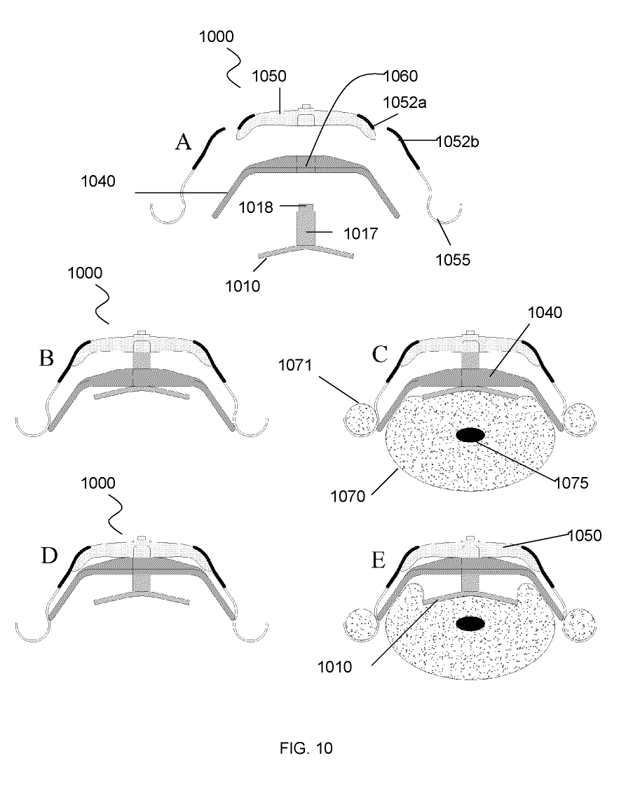

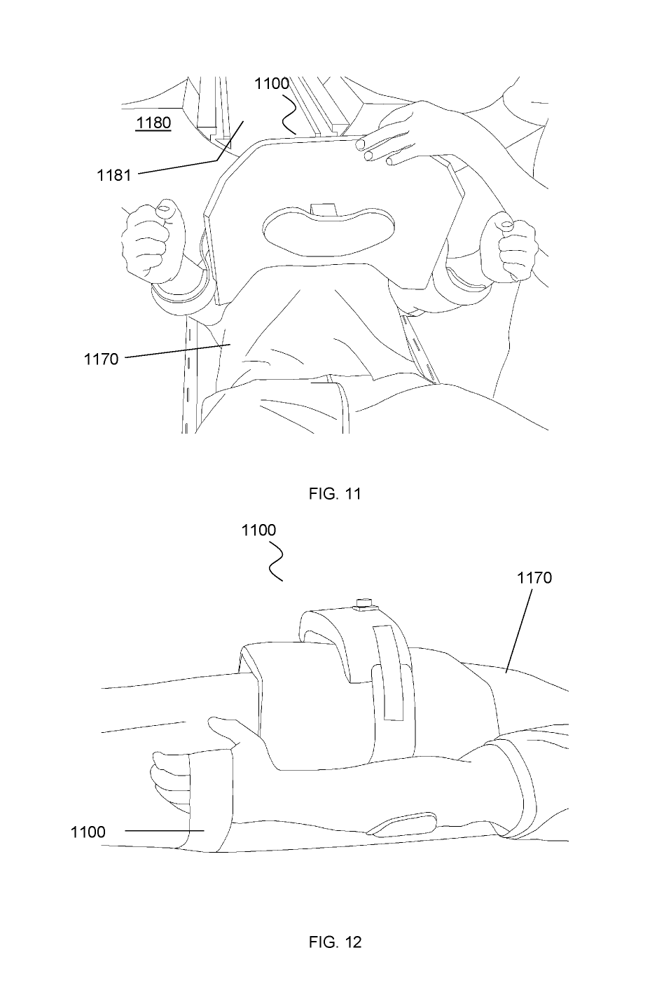

In the embodiment illustrated in FIGS. 8 and 9, the MR apparatus 800 may include one or more members or pedestals 817. In one embodiment, the member or pedestals may constitute an integral extension from the top side 813 opposite to the pressing surface. In another embodiment, the top side may include a well or cavity designed for receiving a connecting member or pedestal. Although not shown in the figures, it should be understood that the one or more members or pedestals may also extend from a lateral side of the pressing surface. The one or more pedestal members 817 may include an end for mating or coupling with the pressing surface 810 and a free end 818. The one or more pedestals 817 may, in one embodiment, take the form of a hollowed housing which may serve to house MR coil components of significant height, such as pre-amplifiers, and it may also provide a cable path for the indenting substrate. Placing components of relatively significant height within the pedestal member may enable a low or thin profile housing for the pressing surface. For example, if the pressing surface is bordered by lateral side walls 115 as shown in FIGS. 1-4, placing the components within the pedestal member may enable a reduced height for the lateral sides. The one or more members or pedestals 817 may also serve as a connector to other devices that may cooperate with the MR apparatus of the present invention, as it will be described herein below, for example the MR apparatus of the present invention may be used in combination with one or more secondary devices having one or more additional coil elements. The pressing surface having a generally low or thin profile may serve to reduce the distance between the exterior tissue of the subject and the one or more secondary devices, as shown in FIG. 10.

In another embodiment of the present invention, the MR apparatus may also include one or more secondary devices having one or more additional coil elements. The secondary device may be used to provide coverage for portions of the subject that are not covered by the pressing surface.

The one or more secondary devices may be fixed to the pressing device, or the one or more secondary devices may be capable of attachment or detachment from the pressing device. FIGS. 8 and 9 illustrate an MR apparatus 800 having a pressing surface 810, a secondary device 840 and a pedestal or member coupling 817 with free end 818. The pedestal member 817 may serve as a connector between the pressing surface 810 and the secondary device or substrate 840. In this embodiment, the secondary device 840 may be provided with a channel 860, which may or may not extend through the width of the secondary device. The outer dimensions of the pedestal member may lie within the channel of the secondary device. FIGS. 6 and 7 c) illustrate a channel 160 of secondary device 140. In this embodiment, the pedestal member 817 may slide inside the channel 860 thereby providing a sliding connection between the pressing surface 810 and the secondary device 840.

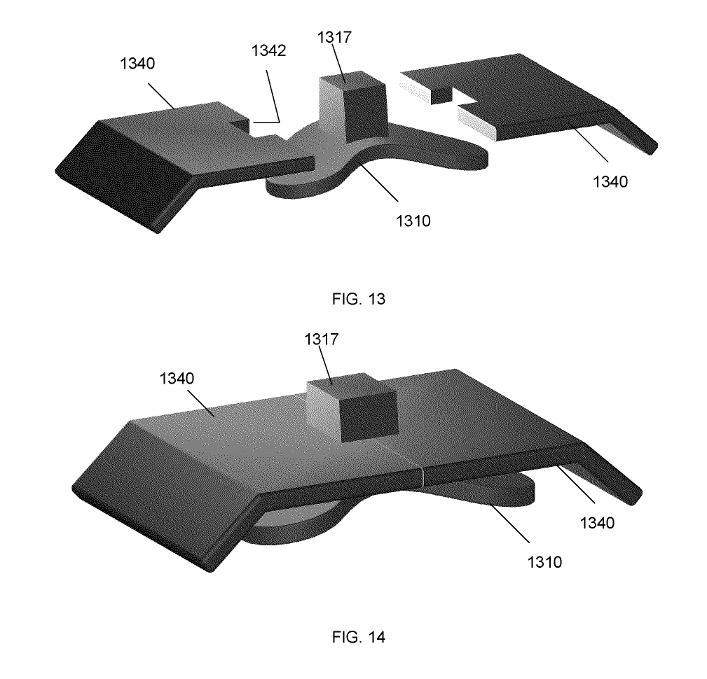

When two or more secondary devices are provided, the secondary devices may be made into modular pieces or modules that fit together or interlock such as to create an engaging means to receive the pedestal or member coupling. FIGS. 13 and 14 illustrates two secondary devices or substrates 1340 provided with cut outs 1342, such as when the secondary devices 1342 are fit together as shown in FIG. 14, the cut outs 1342 create a channel suitable for receiving the member coupling 1317 of the pressing surface 1310.

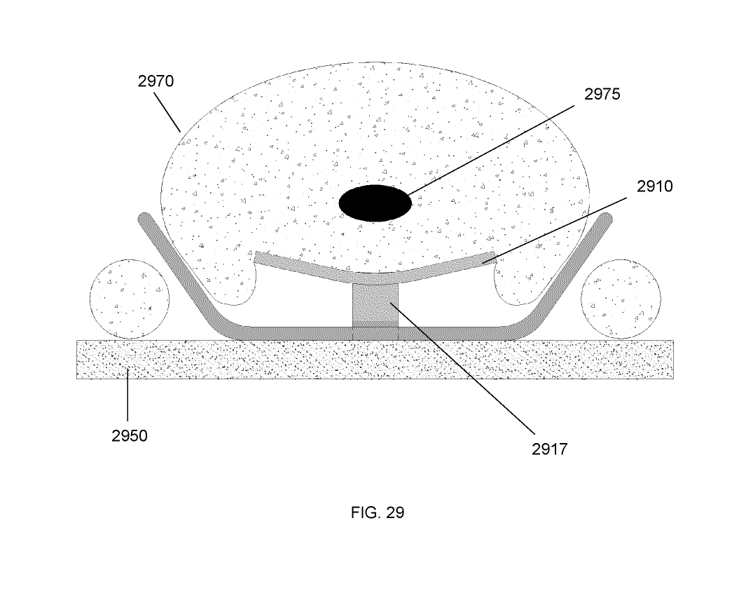

The MR apparatus of the present invention may also be provided with a device for applying physical force to the pressing surface. The device for applying physical force may take the form of arm rests located on the surface of the pressing device or pressing substrate that is not in contact with the exterior tissue of the subject, i.e. the top side. FIGS. 8 and 9 illustrate a device 850 for applying physical force to the pressing surface 810. The MR apparatus 800 includes a pressing surface 810, a secondary device 840, a pedestal member 817 extending from the pressing surface 810 and through a channel 860 on the secondary device 840, and a device 850 for applying physical force connected to the free end of the pedestal member. The device for applying physical force 850 may include limb support 855 configured for receiving the limbs of the subject. In another embodiment, the device for applying physical force may take the form of a weight that can be connected to the MR apparatus. It should be understood that the mass of the pressing surface itself (i.e. without the device for applying physical force) may also provide sufficient weight for applying the physical force. In yet another embodiment, the device for applying physical force may take the form of a driving means, i.e. a motor or leaver system, connected to the MR apparatus. The physical force may also be applied by a person other than the subject being tested. As illustrated in FIG. 29, the subject 2970 being tested may apply the physical force by resting on the pressing surface 2910, so as to bring the pressing surface 2910 closer to the structure of interest 2975.

If one secondary device is to be used, as shown in FIGS. 8 and 9, then the secondary device 840 may have a periphery 841 that is larger than the periphery 822 of the pressing surface 810. If more than one secondary devices are used (not shown in the figures), then the secondary devices may mate to the indenting substrate around the periphery of the pressing surface so as to provide MR coverage beyond the coverage of the pressing surface.

The pressing surface and, if one is provided, the coupling members, may be made of a substantially rigid, non-ferromagnetic material.

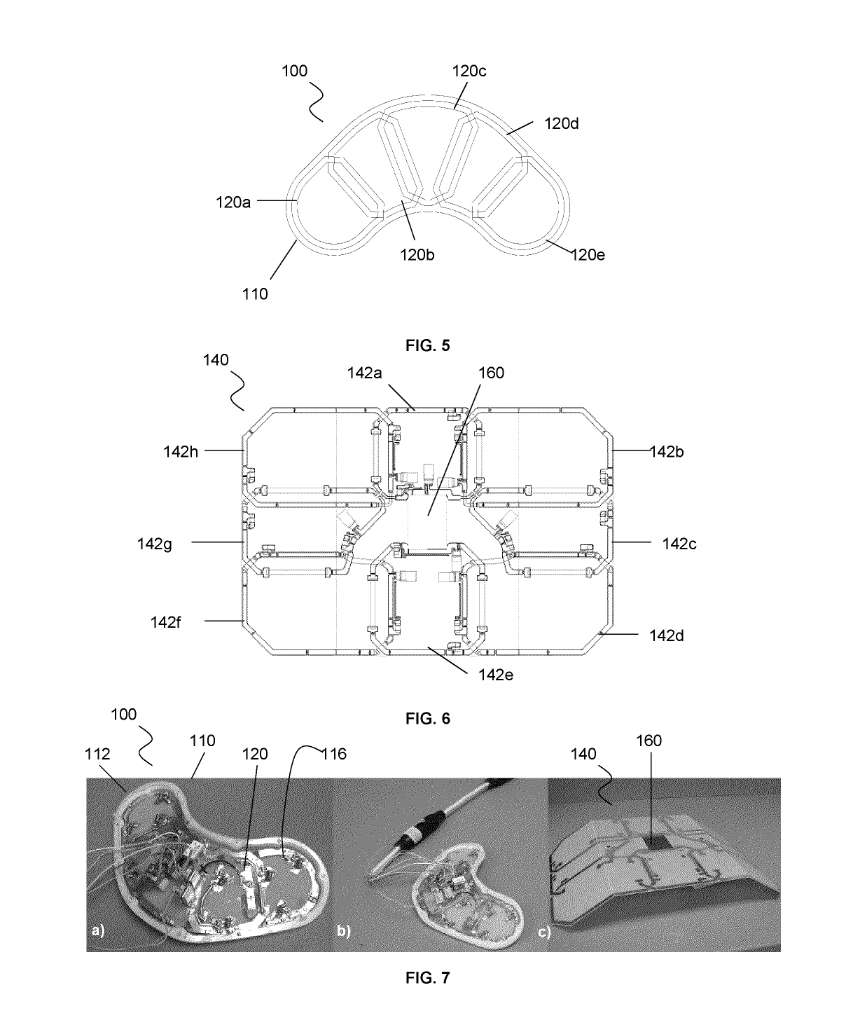

The MR coil elements coupled to the pressing surface or to the secondary device may be any type of suitable coils for MR. The MR coils may be radio frequency (RF) transmitting coils, MR signal receiving coils, gradient coils or any combination thereof. FIG. 5 illustrates a pressing device 110 with 5 MR coil elements 120a-e. FIG. 7a) is a photograph showing the MR coil elements 120 within the casing 116 of the pressing device 110. FIG. 6 illustrates a secondary device 140 housing 8 MR coils 142a-h. It should be understood that the pressing device or the pressing substrate may include more or less than 5 MR coil elements, i.e. 1, 2, 3, 4, 5, 6, 7, 8, 9, 10 coil elements and so on. When the MR apparatus includes a one or more secondary devices, the coil elements coupled to the pressing surface and the secondary device may be laid out so as to (a) reduce electromagnetic coupling of the coil elements of the pressing device or pressing substrate and the coil elements of the secondary devices or secondary substrates, (b) improve signal to noise performance, and (c) improve parallel MRI performance. The MR coil elements may contour the periphery of the surfaces. Each individual coil may take any shape, such as circular, oval, polygon and/or a non-symmetrical shape. The coils may or may not overlap one another.

The pressing surface of the MR apparatus of the present invention may take any suitable shape for pressing or even indenting an exterior tissue of a subject. The design of the pressing surface may take into account the relationship between anatomical morphing capabilities of a tissue to be pressed or indented and the performance of the coil elements enabled by the pressing surface. When the subject is an animal (including humans), the design may also take into account comfort to the subject. FIGS. 1-5, for example, illustrate a design that may be suitable for pressing or indenting a portion of an abdomen, but other designs may be possible. An embodiment of a surface for pressing an abdominal portion of a subject may include a convex curved surface which may be configured to accommodate a curvature of a lower end of a rib cage of the subject, or a curvature of a pubic arch and wings that may generally contour around each side of the abdomen. Referring to FIGS. 1-4, a device 110 suitable for pressing an abdominal portion may take a general bat-like shape having a first wing region 122, a tail region 123, a head region 124, and a second wing region 125. The head region may be defined by a convex shape and the tail region may be defined by a concave shape. It may be desirable that the wing regions 122, 125 be at least substantially symmetric about a midline 126 extending from the head region 124 to the tail region 123. Midline 126 may be situated midway between the two wings 122, 125. In one embodiment, the wing regions 122, 125 may be bent down or curved from the midline 126 as shown in FIGS. 1, 3 and 4 so as to generally conform to the shape of an abdominal portion. If the structure of interest is within a breast, then the pressing surface may take a generally circular, oval shape, or polygon shape (triangular, square, rectangular, pentagon and so forth. In another embodiment, an MR coil may be coated with a suitable coating, such as a suitable insulator. In this embodiment, the MR coil with the coating may be the pressing surface, and used to selectively indent a portion of an exterior tissue.

Although the above description refers to a pressing surface, it should be understood that the MR apparatus of the present invention may also acquire or transmit MR signals from/to a structure of interest without a pressing step. That is, the pressing surface of the MR apparatus of the present invention is suitable for pressing, but it may just be positioned on the exterior tissue of the subject and rest on the exterior tissue of the subject.

FIGS. 5-7 show a coil element construction within a pressing device 110 (FIGS. 5 and 7 a)) and within a secondary device 140 (FIGS. 6 and 7 c)). The pressing device 110 of FIGS. 5 and 7a) is shown as having 5 coils 120a-e. The secondary device of FIGS. 6 and 7 c) is shown as having 8 coil elements 142a-h. As previously stated the pressing surface, as well as the secondary device, may have any number of coils.

The pressing surface or tissue contacting surface or the MR apparatus of the present invention may be sized to cover a portion of a body or body part. The size of the pressing surface or tissue contacting surface may depend on body or body part to be analyzed with the MR apparatus and/or the structure of interest within the body or body part. For example, in the case of the abdomen the size of the pressing surface or tissue contacting surface may range from about 1 cm in diameter to about 20 cm in diameter; for breasts, the pressing surface or contacting surface may range from about 1 cm in diameter to about 10 cm in diameter.

Operation

The MR apparatus of the present invention may be used in methods for recording a MR signal from a structure of interest inside a subject, and/or to transmit a Larmor frequency signal to a structure of interest inside a subject. In one embodiment, a method may begin by providing a MR apparatus of the present invention having one or more MR signal receiving coils and/or one or more coils capable of transmitting a Larmor frequency, and a tissue pressing surface. The design of the MR apparatus to be provided may depend on the structure of interest and the subject. For example, when the structure of interest is inside the abdomen, then an MR apparatus of FIGS. 1-5 may be used.

FIG. 10 illustrates the operation of an MR apparatus of the present invention. With reference to FIG. 10 A, the MR apparatus 1000 to be provided may include an pressing surface 1010 having pedestal member 1017 with free end 1018, a secondary device 1040 with a channel 1060 and a device 1050 for supplying physical force to the pressing surface having limb support 1055. The pressing surface may be mated with the secondary device by the pedestal. The physical force device 1050 may then be connected to the free end 1018 of the pedestal member 1017. The limb support members 1055 may then be attached to the device 1050 for applying the physical force. The limb support member may be coupled or attached to the device 1050 by any suitable connecting means 1052a,b, for example, hook and loop tapes (i.e. Velcro™) FIG. 10 B illustrates the assembled MR apparatus 1000.

With reference to FIG. 10 C, the pressing surface 1010 of the MR apparatus 1000 may be positioned to contact a portion of a subject's 1070 exterior tissue that is relatively near to where the structure of interest 1075 is located inside the subject 1070. The subject 1070 may then rest his/her limbs 1071 in the limb support members 1055. The portion of the subject's exterior tissue may then be exposed to an MR system having a Larmor frequency (not shown in FIG. 10). The MR system may be any suitable MRS or MRI system. With reference to FIG. 10 E the method may continue by applying physical force to the pressing surface 1010 thereby selectively pressing, or indenting, the portion of the subject's 1070 exterior tissue and reducing a distance between the one or more MR signal receiving and/or signal transmitting coils, or gradient coils of the pressing surface 1010 and the structure of interest 1075 inside the subject 1070. The physical force may be applied by the subject by pushing the limb support members down. The means for applying a physical force may also include adding a weight to the MR apparatus, or by using a driving means, or by having a person other than the subject being tested pushing the MR apparatus against the person. In the embodiment shown in FIG. 29, the subject 2970 being tested may rest on the pressing surface 2010 so as to position the pressing surface 2010 in contact with a portion of the subject's exterior tissue that is relatively near to where the structure of interest 2975 is located inside the subject 2970. In one embodiment, the weight of the subject 2970 may serve as the physical force being applied to the pressing surface to selectively press the portion of the subject's exterior tissue and bring the coils of the MR apparatus 2010 closer to the structure of interest 2975. In the embodiment of FIG. 29, the pressing surface 2910 may be positioned on a resting surface 2950, such as a table. The table 2950 may include a receiving means for receiving a member coupling 2917. The receiving means may allow a variation in the movement of the coupling and, by extension, the press 2910, relative to the surface of the table 2950, or the coupling 2917 may be fixed to the table 2950. The pressing of the subject 2970 may be controlled by a number of different ways. For example, the pressing may be controlled by varying the length of the coupling, or by adjusting the height of the press relative to the surface of resting device, or by the subject pushing against the press, or by raising the table or a portion of the table.

While the physical force is applied, the structure of interested may be stimulated at the Larmor frequency of the MR system. Stimulation at the Larmor frequency may be done with the transmitting coils of the MR apparatus of the present invention. The MR signals from the stimulated structure of interest may then be received by the MR signal receiving coils of the MR apparatus of the present invention.

FIG. 11 illustrates the positioning of the MR apparatus 1100 in contact with a subject 1170 prior to moving the subject into the bore 1181 of MR system 1180 and exposing the subject 1170 to the MR system 1180. FIG. 12 illustrates the MR apparatus 1100 positioned in contact with the subject 1170, in this case on top of the subject 1170. As illustrated in FIG. 12, a flexible member, such as belt 1190, may be used to provide leverage for the subject 1170 applying a downward force with the limbs in the limb support 1191.

Different inhale/exhale indentation conditions may be used to improve pressure depth. In one embodiment, the pressing may be carried out as follows:

i) inhale with pressing of the MR apparatus;

ii) holding the press while transmitting a signal at the Larmor frequency to the structure of interest and receiving MR signal from the structure stimulated with the Larmor frequency; and

iii) exhale and release the press.

Another condition may be:

i) inhale ii) press and hold press, iii) exhale and hold breath while holding press, iv) stop holding breath and release press.

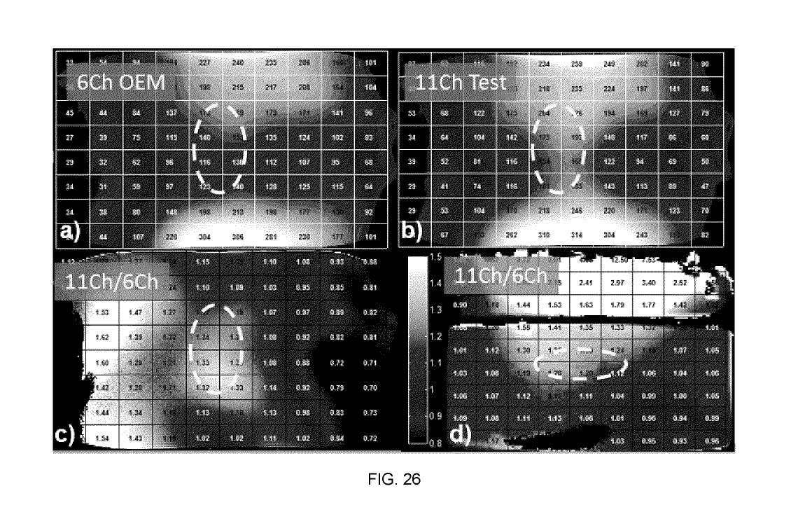

Although the pressing surface of the present invention may be used for pressing the exterior tissue of a subject to bring the MR coils relatively closer to a structure of interest, the MR apparatus of the present invention may also be operated without a pressing step. As shown in FIGS. 26 c) and d) SNR improvement was achieved from the pressing surface of the present invention without a pressing step.

### Advantages

The embodiments of the MR apparatus of the present invention provide the following, non-limiting advantages:

1. Using a pressing surface to reduce the distance between MR coil elements and a target of interest inside a body enables improved SNR behaviour from the coil elements. There may also be a parallel MRI benefit (improved g factor) with pressing or even indenting relative to the same coil un-pressed.

2. Reduce patient discomfort by having the patient control the pressing force.

3. House all electronic components required for the coils within the pressing surface.

4. Pressing surface rigid enough to not deform under the physical force applied by the subject.

5. Safely enclose the components and cables to a level suitable for human REB protocol testing or clinical imaging.

6. Maintain a narrow or thin profile to sit nicely posterior to the larger housing of the secondary device.

7. The narrow or thin profile of the pressing surface enables the secondary device being positioned relatively closer to the subject.

8. Have a variable depth sliding connection with the larger housing of the secondary device.

By limiting the dimensions of the pressing surface to substantially less than the size of the subject, object, or the part thereof for which the MR apparatus of the present invention is designed, the tissue pressed into has a space around the pressing surface, into which to move. This tends to decrease a compression of the tissues, which is an advantage for comfort of the patient and reduces a force required for a particular penetration depth. While some minimal amount of compression may be inevitable with the present invention depending on the tissue being pressed, substantial compression of the tissue is not necessary for the MR coils to approach the structure of interest.

### EXAMPLES

These Examples are described solely for purposes of illustration and are not intended to limit the scope of the invention. Changes in form and substitution of equivalents are contemplated as circumstances may suggest or render expedient.

### Example 1—Simulation Experiments

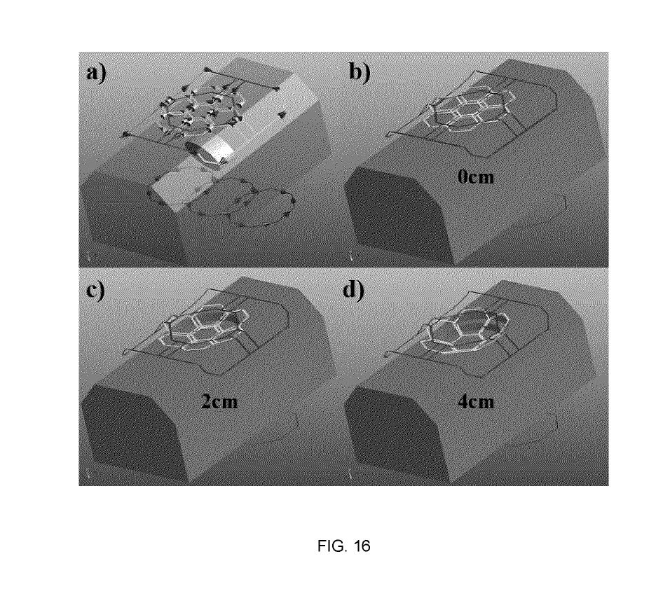

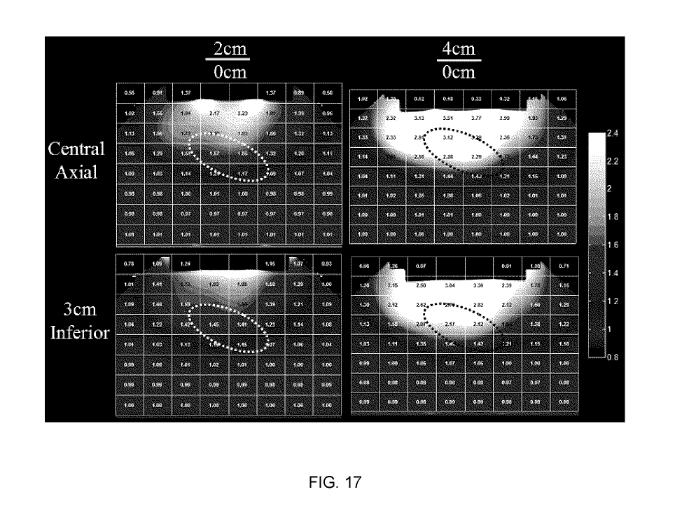

Pressing a subject's exterior tissue to reduce the distance between a structure of interest inside the subject and a MR coil, results in improved MRS/MRI performance. To investigate the viability and performance improvements of this approach, a computational simulation of a viable coil design under the described conditions was conducted. Using a human torso sized phantom with an anatomically placed pancreatic tissue region, a 14Ch coil design was simulated based on 0 cm, 2 cm and 4 cm indentation into the torso model (FIG. 16). With the pancreas in mind, SNR and parallel imaging performance was analyzed for various sagittal, coronal and axial slices of the phantom for each condition.

Relative SNR performance for two different axial slices is shown in FIG. 17. This viability investigation confirmed that both significant SNR and parallel imaging behavior will be achieved by utilizing a coil design capable of indenting abdominal tissue to reduce the distance between the pancreas and the MRI receive coil. Although benefits depend on depth of tissue, the current findings indicate SNR improvements in excess of 100% may be achievable for at least the anterior half of the pancreas.

### Example 2—Design of Abdominal MR Apparatus

An RF coil array design was segmented into a portion (i.e. the pressing device) that will press the abdominal tissue to provide high signal sensitivity to an abdominal structure, such as the pancreas, and a larger portion to sit above (anterior) for providing signal coverage over the entire torso (i.e. the secondary device). This large portion may be used to improve parallel imaging performance of the array. To design the pressing portion of the RF coil array, we had to determine the shape and orientation that will provide the best pressing or indenting structure to design the RF coil in. This presents a relationship between anatomical morphing capabilities of the abdominal region and the coil performance properties enabled by the associated pressing device.

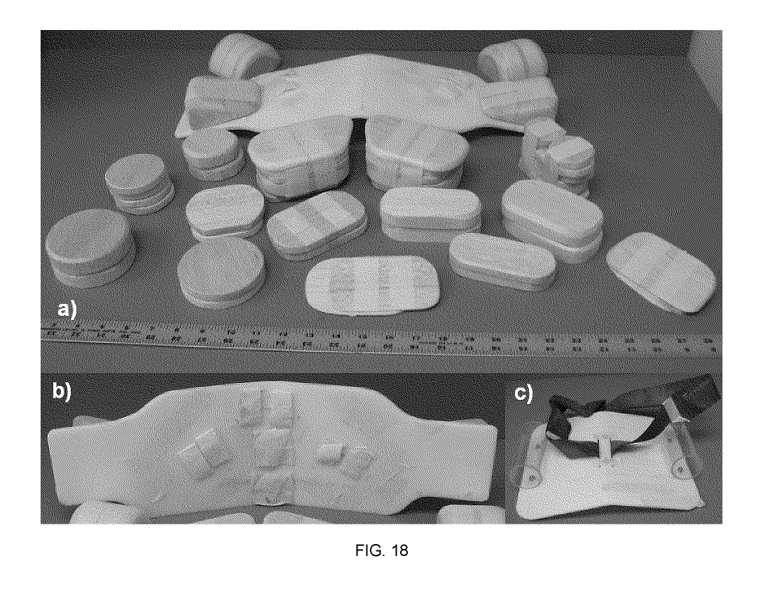

To investigate potential pressing surfaces or devices, a variety of shapes, sizes, orientations and depths of paddles were tested on a group of volunteers. These paddles were made using balsa wood, as shown in FIG. 18. The design process involved evaluating the comfort of various pressing paddle configurations, as well as utilizing MRI data to measure the distance from the pressing pad to the patient's pancreas, to allow determining pad configurations that allow optimal pressing. For safety, an important constraint was that the patient is given complete control over the physical force for pressing and comfort level. Based on leverage, comfort and magnet bore restrictions, the one design approach involved placing arms on top of a white plastic support (shown in FIG. 16a) or pushing arms through a loop in a flexible member, like a belt or strap connected to the top of a other plastic support (shown in FIG. 16c) and then wrapping another belt just below the patient's waist (shown in FIG. 12). Grasping the belt with their hands, the pressing force comes from the patient's mid-arms (elbows) moving downward on mechanical arm pads that transfer the force to the indentation paddle. This also avoids any unwanted patient body positions such as crossing arms to avoid possible MRI complications.

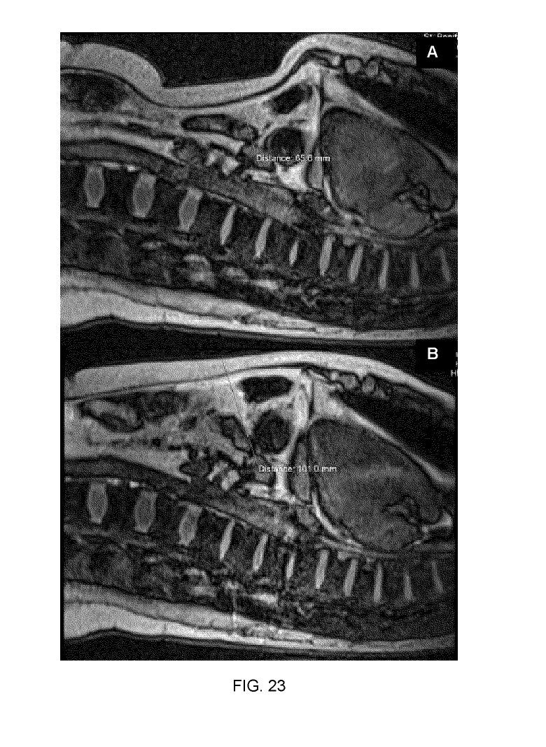

Due to the position of the pancreas, the paddle was designed to be as close as possible to the rib cage. Also, it may be desirable for the pad face to be given a slight superior angle, pointing it superiorly towards the pancreatic region. This angle can be seen in FIG. 23A. Special consideration was given to avoid the xiphoid process. Performance based considerations also included the volume of tissue capable of being displaced, the S-I angle of the pad when indented, and the viability of the indentation pad to extend the coil design to prostate imaging or other internal organs such as kidneys. This evaluation required collecting multiple sets of MRI data from different volunteer patients having various torso sizes and shapes.

### Example 3—Evaluation of Abdominal MR Apparatus

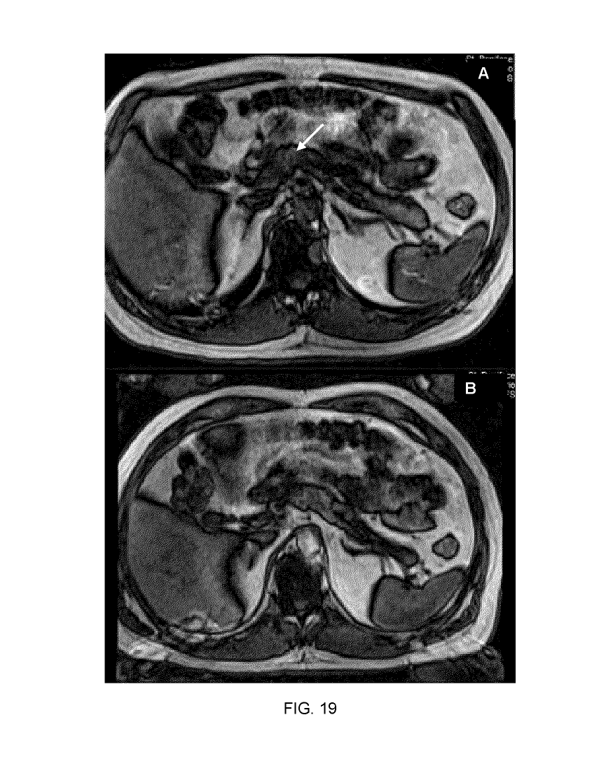

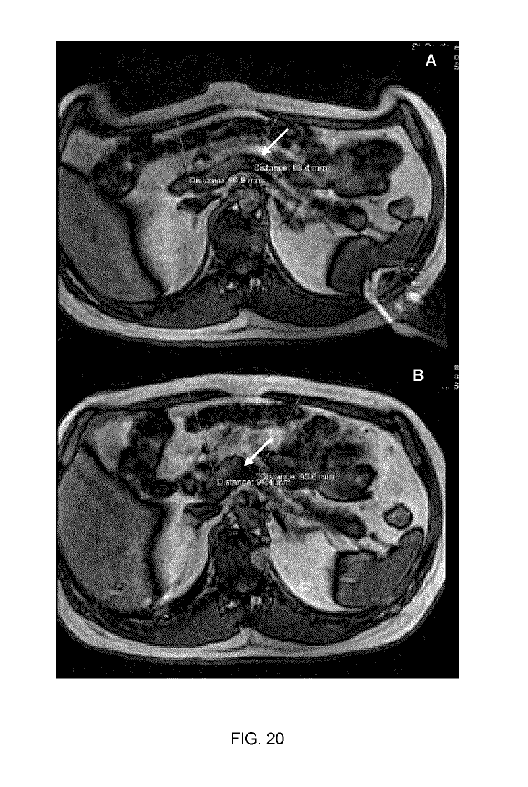

Different inhale/exhale indentation conditions were tested to improve depth of pressure. Also considered was the order of operations (exhale then indent, vs. indent then exhale) to see if pancreas position or orientation changed. Two of the sample patient datasets are shown in FIGS. 19-23.

1. Equipment and Subject

MRI system: Siemens 3T Verio.

Human volunteer: 100 kg healthy adult male.

A variety of shapes, sizes and thicknesses of pressing pads shown in FIG. 16 a).

2. Imaging Sequence

T1 FLASH (Axial)—80 slices (interleaved contrast), FOV 289 mm×360 mm (165×256), 5 mm slice, TR=3.95 ms, TE=1.19 ms and 2.14 ms. [Note that the sequence is numbered based on system tracker]:

2) Inhale with press (axial)

3) Inhale without press (axial)

4) Exhale with press (axial)

5) Exhale without press (axial)

6) Press and then inhale (axial)

7) Inhale with press (sagittal)

8) Inhale without press (sagittal)

Changed Geometry of Indenting Substrate

10) Inhale with press (axial)

12) Inhale without press (axial)

13) Inhale with press (sagittal)

14) Inhale without press (sagittal)

3. Results

Exemplary images of the sequences are displayed in FIGS. 19 and 20.

FIG. 19 illustrate inhale (panel A) vs. exhale (panel B) tracking of the pancreas (white arrow), corresponding to datasets 3 (inhale without press (axial)) and 5 (exhale without press (axial)). An inferior movement of the pancreas from inhale of about 35 mm was observed.

The benefits of pressing with inhale are clearly demonstrated in FIG. 20.

FIG. 20 A corresponds to data set 2 and FIG. 20 B corresponds to dataset 3. The distance between the pressing device and the pancreas (white arrow) in dataset 3 is about 95 mm (note that the pressing device is deforming the anterior tissue by some amount in this data set). The distance between the pressing device and the pancreas (white arrow) in dataset 2 is about 67.6 mm. The distance gain from press and inhale is about 27.4 mm.

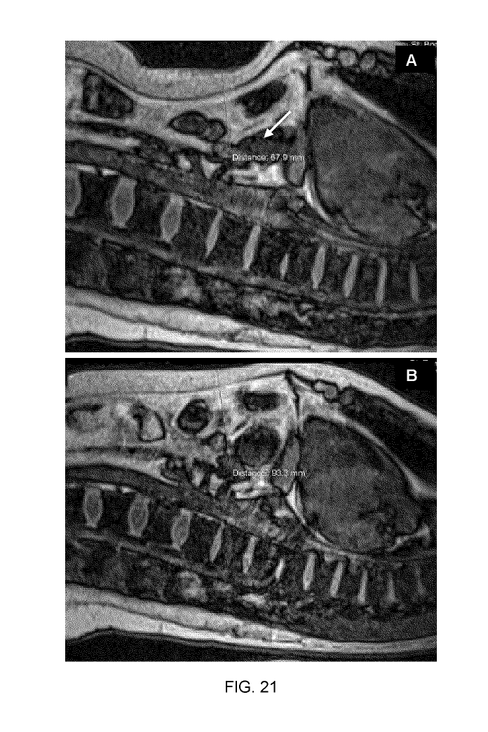

The benefits of pressing are seen in FIG. 21. FIG. 21 A corresponds to data set 7 (inhale with pressing (sagittal)) and FIG. 21 B corresponds to dataset 8 (inhale without pressing (sagittal)). The distance between the pressing device and the pancreas (white arrow) in dataset 7 is about 67.9 mm. The distance between the pressing device and the pancreas in dataset 8 is about 93.3 mm (note that the pressing pad is deforming the anterior tissue by some amount in this data set). The distance gain from pressing and inhale is about 25.4 mm.

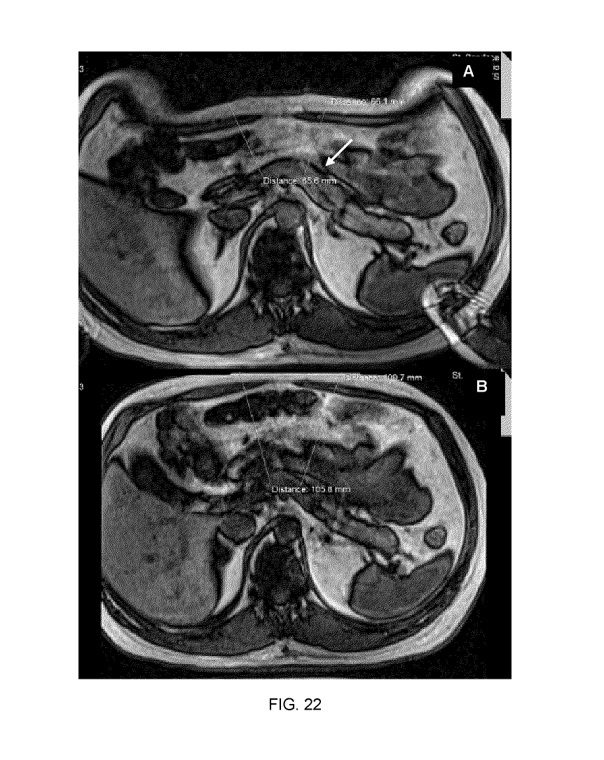

FIGS. 22 and 23 were obtained using a pressing pad different from the one used in FIGS. 20 and 21. FIG. 22 corresponds to dataset 10 and FIG. 20B corresponds to dataset 12. The distance gain from pressing is 107.7 (dataset 12)−65.9 (dataset 10)=41.8 mm. The distance gain from pressing between datasets 13 (FIG. 23A) and dataset 14 (FIG. 23B) is about 35.4 mm.

In conclusion, using the pressing pad of the present invention reduces the distance between the pressing pad and the structure of interest inside the body of a subject. In the case of the pancreas, the reduction in distance may be augmented by having the subject inhale prior to pressing.

### Example 4—Electrical Layout of a Pressing Device for the Pancreas

A printed circuit board (PCB) was engineered to optimally fill the pressing device space. The pressing device was designed to be a 5-channel coil based on the footprint of the pressing surface of the pressing device and the information gathered regarding pancreas region size and expected distance from the pressing surface. Also created a coil layout (secondary device) for an 8 channel array to provide coverage for the remainder of the torso. This pancreas coil design may be placed anterior to the patient. For parallel imaging purposes, an additional posterior coil is desirable. The standard Original Equipment Manufactured (OEM) posterior spine array coil available on the intended MRI system may provide adequate coverage for the torso.

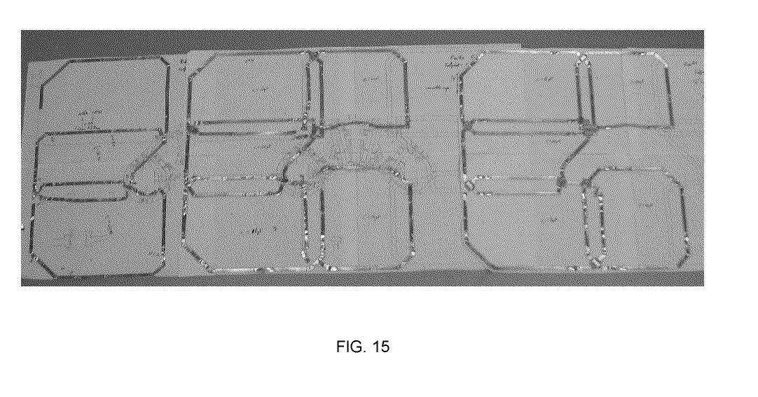

As part of the PCB layout process, multiple iterations of the trace layouts were created to reduce electromagnetic coupling, layout of cable locations, and improve SNR performance. Cable routing and mechanical design are also considered to improve clinical usability. The trace layout iterations are shown for the 8-channel PCB in FIG. 15.

The final PCB's are laid out in computer aided design (CAD) to allow future modifications and potential production engineering. FIGS. 5 and 6 illustrate CAD drawings of the two PCB's generated. The large 8 channel PCB (FIG. 6) was sourced as a non-flexible PCB with three sections, a central section with two smaller side sections that will allow contouring to the body of the patient. Due to the requirement of mating to the curved pressing housing, a flexible PCB was developed for the pressing substrate or device portion of the MR apparatus.

Construction of the 5-channel pressing device array was completed within the mechanical housing, after performance optimization, trouble shooting, investigating cable layout and decoupling. The populated pressing device, with cables for phantom testing is shown in FIGS. 7 a) and b). The PCB layout contained all of the components of significant height near the center. These components may be housed in the pedestal member (see FIG. 8, reference 817). This pedestal may form the mechanical connection between the pressing device and the secondary substrate and also provide a cable path for the cables of indentation substrate. The outer dimensions of the pedestal may lie within the inner channel 160 seen in FIGS. 6 and 7 c). Placing the indentation pad components within the pedestal enables low or thin profile housing for its lateral portions to reduce the distance between the patient and the 8-channel outer secondary device.

Phantom Design and Results

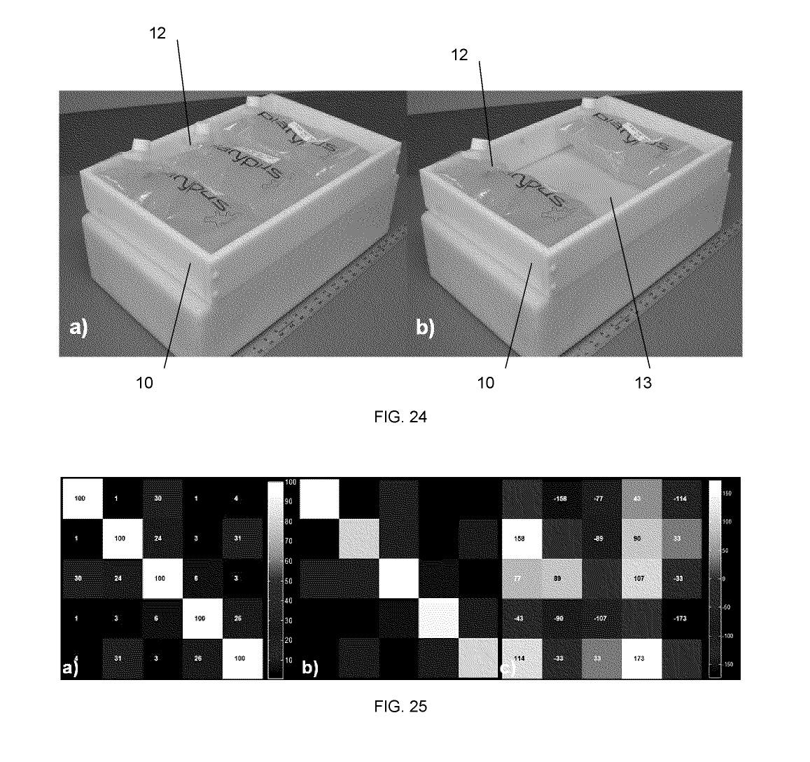

A phantom design that mimics a torso of a human had to represent human torso dimensions, in addition to being capable of quick and reproducible conditions between the pressed and non-pressed state. To achieve this, and with reference to FIG. 24, a rigid housing 10, sized 30 cm×13.5 cm×50 cm (L-R×A-P×S-I), with three 2 L bags 12 of solution placed on top (FIG. 24 a)) or with two 2 L bags (FIG. 24 b)), oriented L-R was used. The three bags of the phantom of FIG. 24 a) fill the entire surface of the rigid housing and it represents a non-pressed state. The two bags of the phantom of FIG. 24 b) are separated by a space 13 and it represents the pressed torso. The bags 12 were contained with a perimeter former to enforce overlapping placement for the non-pressed state. The 2 L bags 12 were chosen as they provide an A-P thickness near 5 cm, which matches our reasonable press distance expectation from the concept development stage. All containers were filled with a solution of 3.5 g/L NaCl and 1.0 g/L CuSO4. The phantom arrangement for the two different states is shown in FIGS. 24 a) and b).

First, we investigated the noise properties of the constructed 5 channel indentation coil shown in FIGS. 7 a) and b) on the phantom, which results are shown in FIG. 25. This step helps confirm proper/optimal function of the coil elements and that all channels are functioning properly with reduced electromagnetic coupling. Next, using the same set-up, we looked at both central axial and 10 cm deep coronal slices under various coil conditions for the non-pressed and pressed phantom states. For full torso coverage, we also utilized the 6 channel Siemens body matrix anterior coil in addition to the 5 channel indentation substrate MR Coil of FIG. 7 a) and b), totaling 11-channels. The 6 channel Siemens Body Matrix coil provides similar performance to what the 8 channel array of the secondary device will contribute. The majority of the experimental SNR coverage within the pancreas region is expected from the pressing device, 5-channel array. Coil performance through experimental SNR maps and experimental SNR gains is shown in FIG. 26 through FIG. 28.

Phantom Performance Discussion and Summary

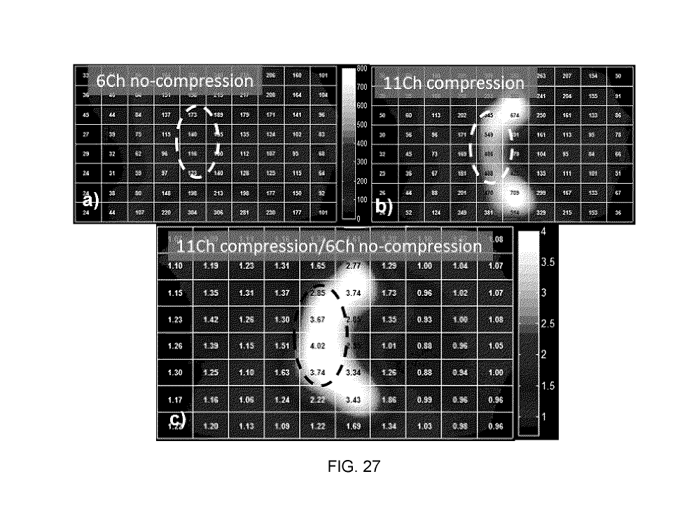

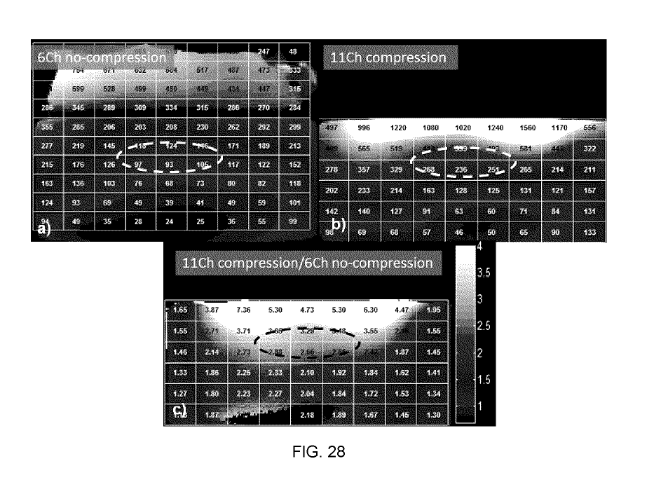

First, we consider the SNR gain from adding the 5Ch pressing device coil array without applying any pressure. This gain in SNR is relative to if the 6 Channel Siemens Body Matrix was used only. From the 10 cm deep coronal and central axial slice data shown in FIG. 24, a 25%-50% SNR gain is shown (FIGS. 24 (c) and 24 (d)) in this situation. The real benefits of this coil design are realized when pressure is applied, as shown in FIGS. 27 and 28 for 10 cm deep coronal and central axial slices respectively. For the coronal slice, the pancreas region displays SNR increases in the range of 200%-300% (FIG. 27 (c)). The central axial slice shows similar SNR gains for the dashed region (FIG. 28 (c)). These SNR performance gains are in excellent agreement with those predicted during the initial simulation based investigations described in Examples 1 and 3.

The mechanical housing for the pedestal portion of the MR apparatus forms a variable separation connection between the 5 channel pressing device and the 8 channel outer device. The rectangular shape allows for two potential pressing device orientations, pointing superior or inferior along the patient's body. This design feature was included for developing this pressing device as both pressing assisted pancreas and prostate array coil.

### Example 5

In Examples 2 and 3 shaped pads were used to study the benefit of pressing. In this example, an MR apparatus of the present invention was used to study the advantages of the MR apparatus of the present invention in improving the signal to noise ratio in MR images.

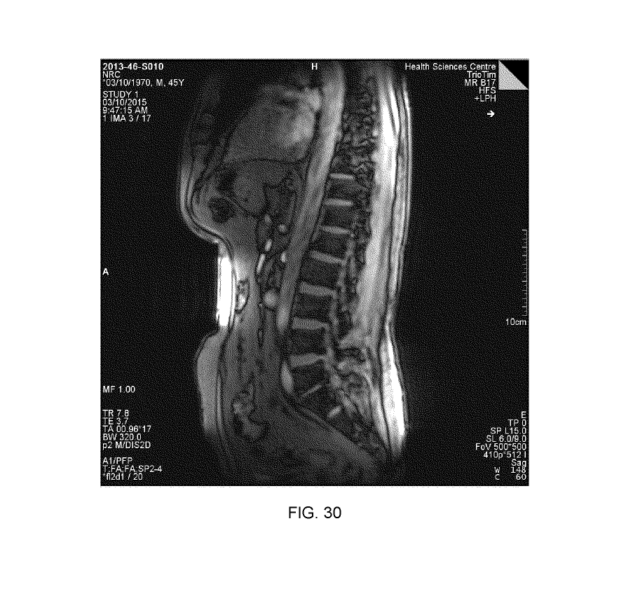

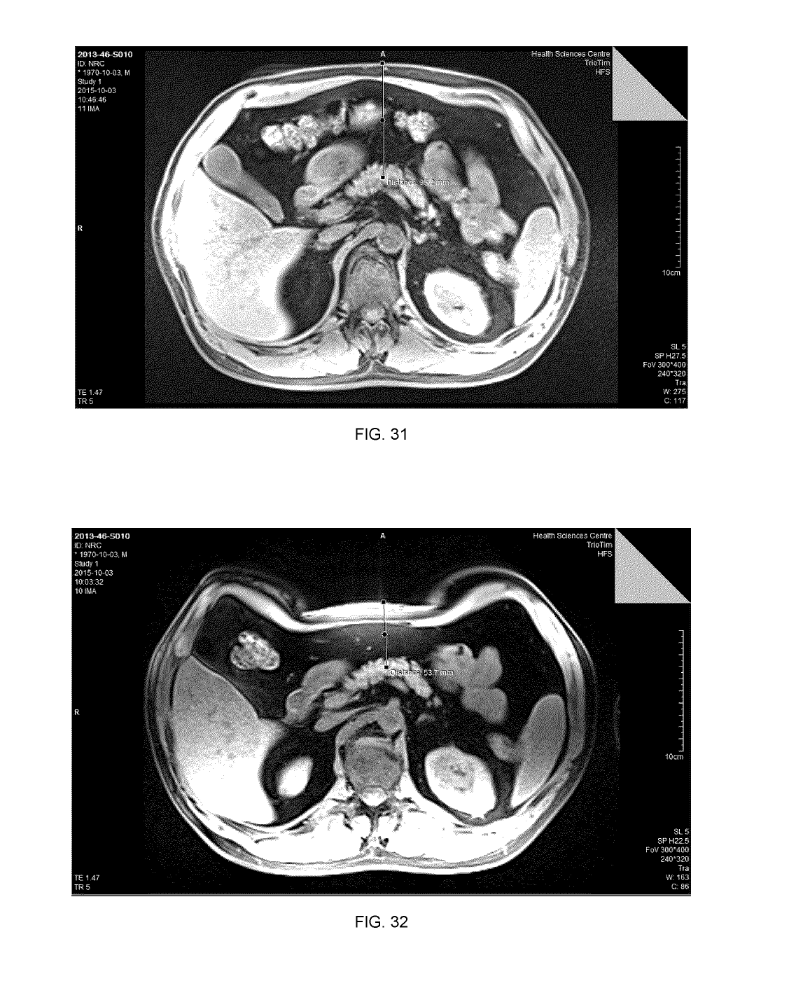

The MR apparatus with RF coil elements shown in FIGS. 10 and 11 was used to obtain images from a healthy human. The images are shown in FIGS. 30-34.

FIG. 30 illustrates a sagittal localizer image (FOV 500 mm×500 mm, Matrix 410×512) using the RF coil shown in FIG. 10 and FIG. 11, with the healthy human subject providing the pressing force described herein, showing indenting into the abdomen. The OEM (Siemens™) spine matrix RF array coil was used under the back of the subject.

FIG. 31 shows a transverse slice from a 3D VIBE MRI (FOV 300 mm×400 mm, Matrix 240×320) using the OEM provided Body Matrix (NON-indenting) RF coil. The OEM spine matrix RF array coil was used under the back of the subject. Depth to the centre of pancreas is 95.2 mm from the surface of abdomen.

FIG. 32 illustrates a transverse slice from a 3D VIBE MRI (FOV 300 mm×400 mm, Matrix 240×320) using the MR apparatus with RF coil shown in FIG. 10 and FIG. 11, with the healthy human subject providing the indenting force described herein. The OEM spine matrix RF array coil was used under the back of the subject. Image was taken following the procedure of taking a deep inhale breath, then pressing, then holding inhale breath for the scan duration of 18 s. Depth to the centre of pancreas is 53.7 mm from the surface of abdomen and the pressing surface having the RF coil. The RF coil is therefore 95.2 mm−53.7 mm=41.5 mm closer to the pancreas. For a pancreas depth of 53.7 mm, RF array elements with size approximately 50-60 mm will provide SNR benefits relative to using the OEM RF array with larger RF array elements (approximately 140 mm in size) with a pancreas depth of 95.2 mm.

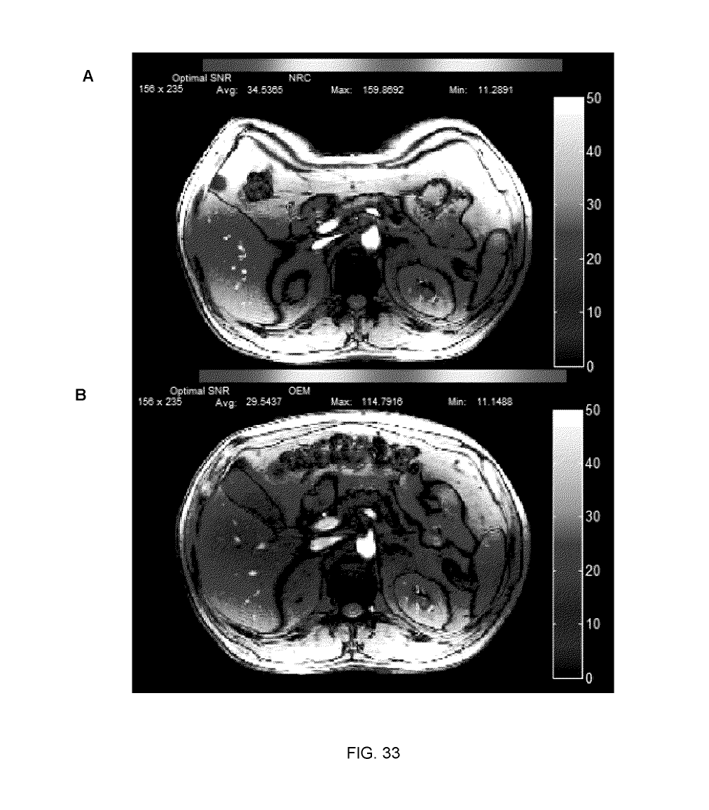

FIG. 33 are SNR maps for the pressing array (panel A) and OEM comparison (panel B). Average SNR in the pancreas region is 33 for the pressing array (panel A) and 16 for the OEM array (panel B), for >2× improved SNR.

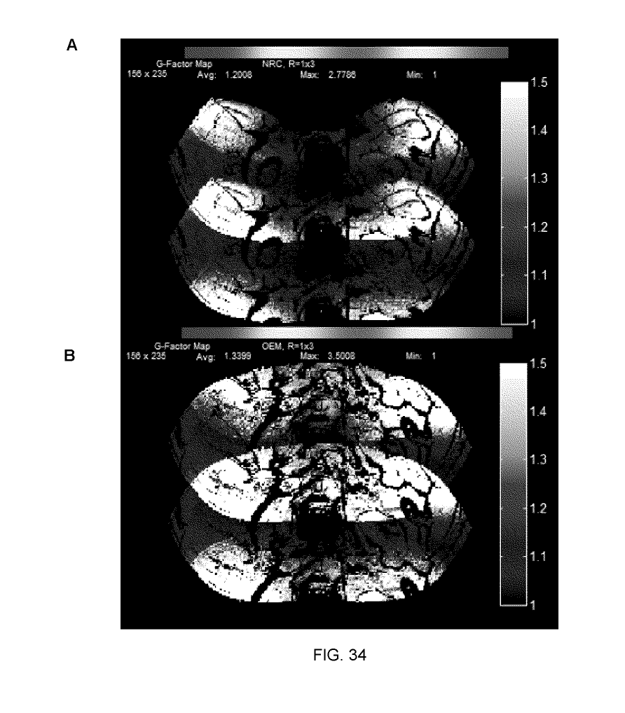

FIG. 34 are g-factor maps for the pressing array (panel A) and OEM comparison (panel B). Average g-factor in the pancreas region is 1 for the pressing array (top) and 1.3 for the OEM array (panel B).

### REFERENCES

[1] J. S. Hyde, A. Jesmanowicz, T. M. Grist, W. Froncisz, J. B. Kneeland, Quadrature Detection Surface Coil, MRM 4, 1987, 179-184.
[2] H. Hricak, S. White, D. Vigneron, J Kurhanewicz, A. Kosco, D. Levin, J. Weiss, P. Narayan, P. R. Carroll, Carcinoma of the Prostate Gland: MR Imaging with Pelvic Phased-Array Coils Versus Integrated Endorectal-Pelvic Phased-Array Coils, Radiology 3, 1994, 703-709.
[3] E. Atalar, P. A. Bottomley, O. Ocali, L. C. L. Correia, M. D. Kelemen, J. A. C. Lima, E. A. Zerhouni, High Resolution Intravascular MRI and MRS by Using a Catheter Receiver Coil, MRM 4, 1996, 596-605.
[4] S. Sammet, Z. Shah, G. Jia, Y. Takayama, R. Jimenez, K. Shah, V. Patel, M. Knopp, Imaging-Pathology Correlation of 3T DCE-MRI Without an Endorectal Coil Used as a Pre-surgical Roadmap for Prostate Cancer Delineation, American Roentgen Ray Society, 2008.

Various modifications may be made in this invention as herein above described, and many apparently widely different embodiments of same made within the spirit and scope of the claims without department from such spirit and scope, it is intended that all matter contained in the accompanying specification shall be interpreted as illustrative only and not in a limiting sense.

What is claimed is:

1. A magnetic resonance (MR) apparatus, the MR apparatus comprising a tissue pressing surface and one or more MR coil elements coupled to the pressing surface, the pressing surface being configured and sized to selectively press a portion of an exterior tissue of a subject when a physical force is applied to the pressing surface thereby reducing a distance between the one or more MR coil elements coupled to the tissue pressing surface and a structure of interest located inside the subject.

2. The MR apparatus of claim 1, wherein the MR apparatus further includes a top surface opposite to the tissue pressing surface, and wherein the one or more MR coil elements are housed in between the tissue pressing surface and the top surface.

3. The MR apparatus of claim 1 or 2, wherein the MR apparatus further comprises one or more secondary devices capable of mating with the pressing surface, each secondary device having one or more additional MR coil elements.

4. The MR apparatus of claim 3, wherein the MR apparatus comprises one secondary device, and wherein the one secondary device has a periphery larger than a periphery of the pressing surface.

5. The MR apparatus of claim 3, wherein the MR apparatus comprises more than one secondary devices, and wherein the more than one secondary devices are capable of mating with the pressing surface around a periphery of the pressing surface.

6. The MR apparatus of any one of claims 2 to 5, wherein the MR apparatus further includes a member coupling having an end attached to the top surface and a free end.

7. The MR apparatus of claim 3, wherein the pressing surface includes a member coupling having an end attached to the pressing surface and a free end, and wherein at least one of the one or more secondary devices includes an engaging means for receiving the free end of the member coupling.

8. The MR apparatus of claim 7, wherein the engaging means comprises a channel extending through the at least one of the one or more secondary devices.

9. The MR apparatus of claim 3, wherein the pressing surface includes a member coupling having an end attached to the top surface and a free end, wherein the MR apparatus comprises two or more secondary devices, and wherein the two or more secondary devices are modular pieces that fit together to create an engaging means for receiving the free end of the member coupling.

10. The MR apparatus of claim 6, 7, or 9, wherein the member coupling includes a hollow interior configured to house electronic components of the one or more coil elements coupled to the pressing surface.

11. The MR apparatus of any one of claims 1-10, wherein the MR apparatus further includes a means for applying a physical force to the pressing surface.

12. The MR apparatus of claim 7, wherein the MR apparatus further includes a means for applying a physical force to the pressing surface, and wherein the means for applying physical force is capable of attachment to the free end of the member coupling.

13. The MR apparatus of claim 11 or 12, wherein the MR apparatus further includes a driving means operatively linked to the means for applying physical force.

14. The MR apparatus of claim 11, 12 or 13, wherein the subject has limbs and the means for applying a physical force to the pressing surface includes limb support configured to receive the limbs of the subject.

15. The MR apparatus of any one of claims 3-14, wherein the subject is an animal and the one or more secondary devices are sized to cover a torso of the animal.

16. The MR apparatus of claim 3, 4, 5, 7, 8 or 9, wherein the additional one or more coil elements of the secondary devices include MR signal receiving coils, RF transmitting coils, gradient coils or any combination thereof.

17. The MR apparatus of claim 3, 4, 5, 7, 8, 9 or 10, wherein the additional one or more coil elements of the one or more secondary devices are laid out within the secondary devices so as to reduce electromagnetic coupling with the one or more coil elements of the pressing surface when the one or more secondary devices are mated with the pressing surface.

18. The MR apparatus of any one of claims 1-10, wherein the subject is a human and the MR apparatus further includes a means for use by the human subject to apply a physical force to the pressing surface, and wherein the means for applying physical force is capable of coupling to the pressing surface.

19. The MR apparatus of claim 18, wherein the means for use by the human subject to apply physical force to the pressing surface includes limb support configured to receive limbs of the human.

20. The MR apparatus of claim 1-19 wherein the one or more coil elements of the pressing surface includes MR signal receiving coils, RF transmitting coil, gradient coils or any combination thereof.

21. The MR apparatus of any one of claims 2-20, wherein the pressing surface is made of a substantially rigid material.

22. The MR apparatus of any one of claims 1-21, wherein the pressing surface has a relatively thin profile.

23. The MR apparatus of any one of claims 1-22, wherein the subject is an animal, and the pressing surface is sized to cover an abdominal portion, a chest portion of, a neck portion or a limb of the animal.

24. The MR apparatus of any one of claims 1-23, wherein the subject is an animal and the structure of interest is an organ or a tumor.

25. The MR apparatus of claim 24, wherein the subject is a human.

26. The MR apparatus of any one of claims 1-25, wherein the subject is a vertebrate and the structure of interest includes an organ of a skeletal system, endocrine system, urogenital apparatus, digestive apparatus, respiratory apparatus, circulatory system, nervous system, synovial fluid, blood and lymph of the vertebrate.

27. The MR apparatus of any one of claims 1-26, wherein the subject is a mammal, and the structure of interest is inside a breast of the mammal.

28. The MR apparatus of any one of claims 1-28, wherein the subject is a human and the pressing surface is configured to press on a breast of the human in the prone position.

29. The MR apparatus of any one of claim 1-12, 16, 17, or 20-22, wherein the subject is a MR phantom and wherein the exterior tissue is an exterior surface of the phantom.

30. The MR apparatus of any one of claim 1-12 16, 17, or 20-22, wherein the subject is a plant, and wherein the exterior tissue is an epidermis of the plant.

31. The MR apparatus of any one of claims 1-28, wherein the subject is an animal, and wherein the exterior tissue is an epidermis of the animal.

32. The MR apparatus of any one of claim 1-12 or 19, wherein the subject is an organ or a tissue biopsy.

33. The MR apparatus of any one of claims 1-28, wherein subject is a vertebrate, and the pressing surface includes a convex curved surface configured to accommodate an inferior curved costal margin of a rib cage of the subject, or configured to accommodate a curvature formed by a pubic bone and both superior pubic rami, or formed to fit inferior to an abdominal pannus over the pubic bone.

34. The MR apparatus of any one of claims 1-33, wherein the tissue pressing surface comprises a substantially rigid material having a substantially thin profile, the pressing surface comprising a first wing region, a tail region, a head region, and a second wing region, the first wing region and second wing region extending longitudinally away from a midline connecting the head and tail regions, the head region being defined by a convex shape and the tail region being defined by a concave shape.

35. The MR apparatus of claim 34, wherein the one or more MR coil elements contour a periphery of the pressing surface.

36. The MR apparatus of claim 34 or 35, wherein the first and second wing regions curve from the midline such that the tissue pressing surface of the MR apparatus is capable of approximately conform to the shape of the exterior tissue of the subject.

37. The MR apparatus of claim 1, wherein the one or more MR coil elements are coated with a suitable coating, and the combination of each of said one or more MR coil elements with the coating constitutes the pressing surface.

38. A method of recording magnetic resonance (MR) signals from a structure of interest inside a subject, the method comprising:
(a) providing a MR apparatus having a tissue pressing surface and one or more MR signal receiving coils coupled to the pressing surface, the tissue pressing surface being configured and sized to selectively press a portion of an exterior tissue of a subject;
(b) positioning the pressing surface in contact with a portion of the subject's exterior tissue that is relatively near to where the structure of interest is located inside the subject;
(c) exposing the portion of the subject's exterior tissue to an MR system having a Larmor frequency;
(d) stimulating the structure of interest at the Larmor frequency of the MR system; and
(e) receiving MR signals from the stimulated structure of interest with the one or more MR receiving coils of the MR apparatus.

39. The method of claim 38, wherein prior to step (d) the method further comprises applying a physical force to the pressing surface thereby selectively pressing the portion of the subject's exterior tissue and reducing a distance between the one or more MR signal receiving coils of the MR apparatus and the structure of interest inside the subject.

40. The method of claim 38 or 39, wherein the MR apparatus further includes one or more transmitting coils capable of transmitting signals at the Larmor frequency of the MR system, and wherein the stimulation of the structure of interest is done with the transmitting coils of the MR apparatus.

41. The method of claim 38, 39 or 40, wherein the MR apparatus further includes one or more gradient coils, and wherein the method further includes performing variations in a main magnetic field (Bo) of the MR system with the gradient coils.

42. The method of claim 38, 39, 40 or 41, wherein the MR apparatus is the MR apparatus of any one of claims 1-37.

43. A method of transmitting signals at the Larmor frequency of a magnetic resonance (MR) system to a structure of interest inside a subject, the method comprising:
(a) providing a MR apparatus having a tissue pressing surface and one or more transmitting coils coupled to the pressing surface, the tissue pressing surface being configured and sized to selectively press a portion of an exterior tissue of a subject;
(b) positioning the pressing surface in contact with a portion of the subject's exterior tissue that is relatively near to where the structure of interest is located inside the subject;
(c) exposing the portion of the subject's exterior tissue to an MR system having a Larmor frequency; and
(d) transmitting the signal at the Larmor frequency with the one or more transmitting coils thereby stimulating the structure of interest.

44. The method of claim 43, wherein prior to step (c) the method further comprises applying a physical force to the pressing surface thereby selectively pressing the portion of the exterior tissue and reducing a distance between the one or more transmitting coils and the structure of interest.

45. The method of claim 43 or 44, wherein the MR apparatus further includes MR signal receiving coils, and wherein the method further includes the step of receiving MR signals from the stimulated structure of interest with the one or more MR signal receiving coils of the MR apparatus.

46. The method of claim 43, 44 or 45, wherein the MR apparatus further includes one or more gradient coils, and wherein the method further includes performing variations in a main magnetic field (Bo) of the MR system with the gradient coils.

47. The method of claim 43, 44, 45 or 46, wherein the MR apparatus is the MR apparatus of any one of claims 1-37.

48. The method of any one of claim 39 or 44, wherein the subject is a human and wherein the step of applying the physical force comprises:
(i) having the subject produce an inhalation stroke;
(ii) applying the physical force to the pressing surface during the inhalation stroke;
(iii) holding the pressing surface for a sufficient amount of time while the subject keeps the inhalation stroke;
(iv) having the subject exhale and release the pressing surface; and
(v) repeating sub-steps (i)-(v) as necessary.

49. A use of a magnetic resonance (MR) apparatus having a tissue pressing surface and one or more MR coil elements coupled to the pressing surface to record, transmit or to record and transmit MR signals form a structure of interest inside a subject.

50. The use of claim 49, wherein the MR apparatus is the MR apparatus of any of claims 1-37.

51. A magnetic resonance (MR) apparatus, the MR apparatus comprising a surface having one or more MR coil elements, the surface comprising a first wing region, a tail region, a head region, and a second wing region, the first wing region and second wing region extending longitudinally away from a midline connecting the head and tail regions, the head region being defined by a convex shape and the tail region being defined by a concave shape.

52. The MR apparatus of claim 51, wherein the one or more MR coil elements contour a periphery of the surface.

53. The MR apparatus of claim 51 or 52, wherein the first and second wing regions curve from the midline such that the surface of the MR apparatus is capable of approximately conforming to a shape of a subject or body part.

54. A combination MR apparatus and resting device, wherein the MR apparatus comprises a tissue pressing surface and one or more MR coil elements coupled to the pressing surface, the pressing surface being configured and sized to selectively press on a portion of an exterior tissue of a subject so as to reduce a distance between the one or more MR coil elements and a structure of interest located inside the subject, the pressing surface having a tissue contacting side and another side for contacting the resting device.

55. A magnetic resonance (MR) apparatus, the MR apparatus comprising a tissue pressing surface and one or more MR coil elements coupled to the pressing surface, the pressing surface being configured and sized to selectively press a portion of an exterior tissue of a body part an animal when a physical force is applied to the pressing surface thereby reducing a distance between the one or more MR coil elements coupled to the pressing surface and a structure of interest located inside the subject, wherein the size of the portion of the exterior tissue ranges between about 1/20 to about ⅔ of the exterior tissue of the body part.

56. The MR coil of claim 55, wherein the body part is an abdomen, and the size of the portion ranges between 1/20 to about ⅔ of the exterior tissue of the abdomen.

57. The MR coil of claim 55, wherein the body part is a breast, and the size of the portion ranges between 1/20 to about ½ of the exterior tissue of the breast.
